(19) United States
(12) Patent Application Publication
(10) Pub. No.: US 2004/0002740 A1
(43) Pub. Date: Jan. 1, 2004

(54) System and method for forming a non-ablative cardiac conduction block

(75) Inventor: Randall J. Lee, Hillsborough, CA (US)

Correspondence Address: JOHN P. O'BANION, O'BANION & RITCHEY LLP, 400 CAPITOL MALL SUITE 1550, SACRAMENTO, CA, 95814, US

(73) Assignee: THE REGENTS OF THE UNIVERSITY OF CALIFORNIA

(21) Appl. No.: 10/434,419

(22) Filed: May 7, 2003

Related U.S. Application Data
(63) Continuation-in-part of application No. 10/329,295, filed on Dec. 23, 2002.
(63) Continuation-in-part of application No. 10/349,323, filed on Jan. 21, 2003.
(60) Provisional application No. 60/429,914, filed on Nov. 29, 2002.
(60) Provisional application No. 60/431,287, filed on Dec. 6, 2002.
(60) Provisional application No. 60/379,140, filed on May 8, 2002.
(60) Provisional application No. 60/426,058, filed on Nov. 13, 2002.

(51) Int. Cl. A61N 1/36

(52) U.S. Cl. 607/9

(57) ABSTRACT

A system forms a conduction block in a regions of cardiac tissue at a location associated with a cardiac arrhythmia by delivering a material that is non-ablative into the region. The material include living cells that do not form sufficient gap-junctions with cardiomyocytes to conduct, e.g. myoblasts, stem cells, or fibroblasts. The material may be a non-living agent, such as a polymer agent, e.g. fibrin glue agent or collagen agent. The material may be a combination of living and non-living material that enhances the cellular conduction block. A contact member delivers the material over a patterned region of tissue, such as arcuate, linear, or circumferential patterns. The contact member may include an expandable member or balloon. A guidewire may be used for delivery. Cells used may be autologous, prepared for injection with a kit. Conduction blocks are thus formed without substantially ablating cardiac tissue in the region.

### BACKGROUND OF THE INVENTION

1. Field of the Invention

This invention pertains generally to systems and methods for treating medical conditions associated with the heart, and more particularly to surgical devices and procedures for forming conduction blocks at locations associated with the heart that include cardiac tissue.

2. Description of Related Art

Cellular therapy for treating cardiac conditions has been the topic of significant research and development in recent years, generally for the purpose of increasing cardiac conduction or function. In fact, certain types of injected cells have been observed to couple poorly with indigenous cardiac cell tissues, and various prior disclosures have cited a related decrease in conduction transmission as a significant obstacle to the intended cellular therapy. Some disclosures have cited a desire to in fact modify the properties of injected cells to increase the cardiac tissue coupling for enhanced conduction or contractility.

Tissue engineering techniques utilizing skeletal myoblast transplantation for myocardial repair has in particular gained increased attention with the demonstration that skeletal myoblasts survive and form contractile myofibers in normal and injured myocardium. However, the emphasis of myocardial repair has focused on the preservation of myocardial contractility with little attention given to the effects of tissue engineering on cardiac conduction or effects on cardiac arrhythmias.

In addition, according to previous disclosures skeletal muscle cells may be initially injected as myoblast and thereafter differentiate into myotubes/myofibers. The conduction properties of myoblasts and myotubes are significantly different. Additionally, depending on how old the myoblasts are, they can vary in conduction properties. Therefore, following the injection of certain preparations of myoblasts, a heterogeneous milieu of cells may result which can produce unpredictable insulation results. However, the use of myoblast injections for creation of conduction blocks to treat arrhythmias should nevertheless be effective.

Cardiac arrhythmias are abnormal conditions associated with the various chambers and other structures of the heart, and are typically treated by drug therapy, ablation, defibrillation or pacing.

Cardiac arrhythmias are the leading cause of morbidity and mortality in the United States. In fact, it is believed that about 60% risk of all cardiac deaths are related to malignant ventricular arrhythmias. Atrial fibrillation (AF) is the most frequently occurring sustained cardiac arrhythmia, particularly among the elderly and patients with organic heart disease; and is one of the fastest growing segments of cardiovascular disease in the U.S. Conventional therapies center around ablation (destruction) of the aberrant conduction pathways, though often such pathways are observed to recur at a later date. Implantation of defibrillators and pacemakers are effective but are fraught with failure, high costs, and often undesirable side effects.

The mechanical methods or implantation of pacemakers and/or defibrillators, generally attempt to re-create normal conduction in the heart and fix the initial disturbance. The goal of such conventional therapy is to enhance the normal physiologic process of the normal heart conduction moving from cell to cell, from the SA node to AV node from the atrium to the ventricles. This cardiomyocyte to cardiomyocyte communication and conduction occurs through electromechanical coupling. This coupling is done by intercalated disks composed of adherens and gap junctions. Connexin 43 (Cx43) is the major gap junction protein in the ventricular cardiomyocyte while N-cadherin is the major adherent junction protein. Both are required to synchronize the electrical mechanical communication.

Ablation is generally a treatment technique intended to create conduction blocks to intervene and stop aberrant conduction pathways that otherwise disturb the normal cardiac cycle. Typical ablation technology for forming conduction blocks uses systems and methods designed to kill tissue at the arrhythmogenic source or along an aberrant, cascading conductive pathway, such as by applying energy to destroy cells via hyperthermia such as with electrical current (e.g. radiofrequency or “RF” current), ultrasound, microwave, or laser energy, or via hypothermia using cryotherapy, or chemical ablation such as destructive ethanol delivery to cardiac tissue. Despite the significant benefits and successful treatments that have been observed by creating conduction blocks using various of these techniques, each is associated with certain adverse consequences. For example, ablative hyperthermia or other modes causing necrosis have been observed to result in scarring, thrombosis, collagen shrinkage, and undesired structural damage to deeper tissues.

Atrial fibrillation (AF) is the most common cardiac arrhythmia, effecting approximately 0.4% of the general population and 10% of persons over the age of 65 years of age. AF occurs in as many as 50% of patients undergoing cardiac operations. Patients with chronic AF have symptomatic tachycardia or low cardiac output and have a 5-10% risk of thromboembolic complications/events. A common treatment for AF is cardioversion, alone or in combination with anti-arrhythmic therapy, to restore sinus rhythm. Recurrence rates after such therapy as high as 75% have been reported. Pharmacologic therapy is associated with adverse effects in a significant proportion of patients with AF.

Other more current methods of treating atrial fibrillation include either through a surgical approach, or by use of various forms of energy to ablate conduction to electrically isolate discrete atrial regions. Current methods of ablation procedures have a high rate of re-occurrence and hold high complication rates.

More specifically, ablation devices and methods have been used in order to form conduction blocks as curative or prophylactic measures specifically to treat atrial fibrillation. However, side effects of such approach, including for example thrombus formation along the endocardial surface where the ablation energy is delivered, are in particular concerning in chambers such as the left atrium in particular where thromboembolisms may lead to downstream complications including stroke. Notwithstanding such side effects, ablation devices and systems for atrial fibrillation remains the focus of substantial research and commercial efforts in view of the substantial prevalence and harm from this dangerous medical condition.

There is therefore a need for improved systems and methods for treating cardiac arrhythmias without the complications and risk factors of other previously disclosed therapies.

There is in particular a need for improved systems and methods for forming conduction blocks at locations along cardiac tissue structures without substantially ablating cardiac tissue.

### BRIEF SUMMARY OF THE INVENTION

It is an object of the invention to treat cardiac arrhythmias by forming conduction blocks without substantially ablating cardiac tissue.

It is also an object of the invention to treat cardiac arrhythmias by forming a conduction block without requiring hyperthermia or hypothermia treatment of cardiac tissue.

It is also an object of the invention to treat cardiac arrhythmias without requiring direct surgery techniques.

It is a further object of the invention to treat cardiac arrhythmias using less invasive or minimally invasive systems and methods.

Accordingly, one aspect of the invention is a system for treating a cardiac arrhythmia in a heart of a patient that includes a cardiac delivery system coupled to a source of material that is substantially non-ablative with respect to cardiac cells. The cardiac delivery system is adapted to deliver a volume of the material from the source to a location associated with the patient's heart that includes cardiac cells such that the material is adapted to form a substantially non-ablative conduction block at the location.

In one mode of this aspect, the material is a living material, which in a highly beneficial embodiment is living cells. According to a further beneficial variation of such embodiment, the living cells are myoblasts, such as skeletal myoblasts.

In another mode, the material is a non-living material, which in a highly beneficial embodiment is a polymer agent, and in another variation is collagen or a precursor or analog or derivative thereof.

According to a further beneficial variation of this embodiment, the polymer agent forms a fibrin glue. In an additional feature with respect to this variation, the source of material may therefore include a first source of a first precursor material and a second source of a second precursor material. The cardiac delivery system is adapted to couple to the first and second sources of first and second precursor materials, respectively, and the first and second precursor materials are adapted to be mixed to form fibrin glue that forms the conduction block at the location. In still a further feature, the cardiac delivery system may be in particular adapted to mix the first and second precursor materials prior to delivery to the location. Alternatively, the cardiac delivery system can be adapted to deliver the first and second precursor materials to the location separately such that they are mixed at the location.

According to another mode, the material of the source is adapted to be delivered by the cardiac delivery system into an extracellular matrix between cardiac cells at the location. In one embodiment of this mode, the material is adapted to intervene with gap-junctions between cardiac cells at the location.

According to still a further mode, the cardiac delivery system is adapted to deliver the material to the location along a ventricle wall of a ventricle in the patient's heart.

In another mode, the cardiac delivery system is adapted to deliver the material to the location along an atrial wall of an atrium in the patient's heart.

In still another mode, the cardiac delivery system is adapted to deliver the material to the location where a pulmonary vein extends from an atrium in the patient's heart, such as at the pulmonary vein ostium, or at locations where cardiac tissue extends into pulmonary veins along the pulmonary vein wall or immediately surrounding the pulmonary vein along the posterior atrial wall.

In one further embodiment of this mode, the cardiac delivery system is adapted to deliver the material along a circumferential region of tissue at the location.

According to one variation of this embodiment, the cardiac delivery system includes an expandable member that is adapted to engage the circumferential region of tissue. Such expandable member in according to one beneficial feature may be an inflatable balloon. In a further feature, the cardiac delivery system is adapted to deliver the material to the circumferential region of tissue when the circumferential region of tissue is engaged by the inflatable balloon. According to another feature of this expandable member variation, the cardiac delivery system further includes at least one needle cooperating with the expandable member. The cardiac delivery system according to this feature is configured to fluidly couple the at least one needle to the source of material and to deliver the material to the location via the needle.

According to still a further mode of this aspect, the material of the source includes living cells in combination with a second material that is non-living and that is adapted to enhance formation of the conduction block. In one highly beneficial embodiment of this mode, the second material is a polymer agent, which in one beneficial variation forms a fibrin glue that is adapted to form the conduction block. In another embodiment, the second material is collagen or a precursor or analog or derivative thereof.

In another embodiment of this mode, the second material is adapted to enhance retention of the living cells at the location. In still another embodiment, the second material is adapted to intervene at gap-junctions between adjacent cells at the location.

Another aspect of the invention is a system for treating a cardiac arrhythmia in a heart of a patient that includes a cardiac delivery system that cooperates with means for forming a conduction block at a location associated with the patient's heart that includes cardiac cells and such that cardiac cells are not substantially ablated, or that otherwise includes delivering a material to the region that is characterized as being substantially non-ablative to cardiac cells.

In one embodiment of this mode, the material of the source that is adapted to form the substantially non-ablative conduction block is a living material, which in one highly beneficial variation includes cells, which cells in a further feature may be myoblasts such as skeletal myoblasts.

In another embodiment of this mode, the material of the source that is adapted to form the substantially non-ablative conduction block is non-living material, which in one highly beneficial variation is a polymer agent, and which polymer agent in a further beneficial feature may be a fibrin glue agent, such as the type formed by first and second precursor materials. Further to this latter feature, the source of material may therefore include first and second substantially isolated sources of first and second precursor materials, respectively, that are adapted to be mixed to form fibrin glue which forms the conduction block at the location. In another variation, the material is collagen or precursor or analog or derivative thereof.

According to another mode, the means for forming a conduction block includes means for forming a substantially circumferential conduction block along a circumferential region of tissue at a location where a pulmonary vein extends from an atrium. In one embodiment of this mode, the means for forming the substantially circumferential conduction block includes means for delivering a material to the circumferential region of tissue that is substantially non-ablative with respect to cardiac cells but that forms the conduction block.

According to yet another mode, the cardiac delivery system includes means for locating the location as a region associated with the cardiac arrhythmia. This means for locating the location according to one embodiment of this mode includes an electrode that is adapted to couple to a monitoring system for mapping electrical conduction in the heart.

According to still a further mode, the means for forming the conduction block comprises means for physically separating cardiac cells at the location.

Another aspect of the invention is a method for treating a cardiac arrhythmia in a heart of a patient by forming a conduction block at a location associated with the patient's heart that includes cardiac cells. Further to this method, the conduction block is formed by delivering a material to the location and without substantially ablating cardiac cells.

According to one mode of this aspect, the conduction block is formed by delivering a non-living material to the location that is substantially non-ablative with respect to cardiac cells. In one embodiment of this mode, the material forms the conduction block by intervening with gap-junctions of cardiac tissue with the material.

In another embodiment of this mode, the conduction block is formed by delivering a polymer to the location, which polymer agent may be for example a fibrin glue agent. According to one variation of this mode, the polymer delivery further includes mixing first and second precursor materials within the body of the patient to form the polymer in vivo.

In another embodiment of this mode, the conduction block is formed by delivering a collagen material to the location, or precursor or analog or derivative thereof.

According to another mode of this method, the conduction block is formed by delivering a living material to the location, such as in a highly beneficial embodiment living cells. In a further variation, the living cells being delivered are myoblasts.

According to yet another mode of this method, the region to which the material is being delivered is located along a ventricular wall of a ventricle of the patient's heart.

In another mode, the region to which the material is being delivered is located along an atrial wall of an atrium of the patient's heart.

Another aspect of the invention is a method for treating a cardiac arrhythmia in a heart of a patient by forming a conduction block at a location associated with the patient's heart that includes cardiac cells by delivering living cells to the location. In one highly beneficial mode of this aspect, the conduction block is formed by delivering myoblasts to the location. In another mode, the conduction block is formed by delivering living cells and a second material that is adapted to enhance formation of the conduction block than if the cells were delivered without the second material. In one embodiment, the second material enhances retention of the living cells at the location. In another embodiment, the second material intervenes at gap junctions between cells.

In another embodiment, the second material provides for a physical separation between cells at the location.

In another embodiment the second material is collagen or precursor or analog or derivative thereof.

Another aspect of the invention is a system for treating a cardiac arrhythmia in a heart of a patient that includes a cardiac delivery system that is coupled to an injectable polymer agent. The cardiac delivery system is adapted to deliver the injectable polymer agent to a location associated with the patient's heart that includes cardiac cells.

In one mode, the cardiac delivery system coupled to the injectable polymer is not coupled to a source of living cells.

In another mode of this aspect, the cardiac delivery system is adapted to provide intracardiac delivery of the injectable polymer agent to the location via at least one of the cardiac chambers.

In another mode, the injectable polymer agent is a fibrin glue agent.

In another mode, the injectable polymer agent includes first and second precursor materials that are adapted to be mixed to form a polymer. Further to this mode, in one embodiment the cardiac delivery system is adapted to mix the first and second precursor materials before delivering a polymer formed thereby to the location. In another embodiment, the cardiac delivery system is adapted to deliver the first and second precursor materials to the location separately such that they mix and form the polymer at the location.

In another mode of this aspect, the cardiac delivery system includes at least one needle that is used to deliver the injectable polymer agent.

In another mode, the cardiac delivery system includes a catheter having an elongate body with a proximal and distal end portions and at least one lumen extending between a proximal port located along the proximal end portion and a distal port located along the distal end portion. The proximal port is adapted to couple to a source that contains at least a part of the injectable polymer agent.

Further to this mode, in one embodiment the catheter further includes at least one electrode located along the distal end portion. The electrode is adapted to be coupled to a monitoring system to monitor electrical signals in the heart via the electrode so as to identify the location for delivery of the injectable polymer agent to thereby form the conduction block.

Another aspect of the invention is a system for treating a cardiac arrhythmia in a heart of a patient that includes a delivery system that is coupled to a source of injectable material that includes collagen or a precursor or analog or derivative thereof. The delivery system is adapted to deliver the injectable material to a location associated with the patient's heart that includes cardiac cells.

Another aspect of the invention is a method for treating a medical condition associated with a heart of a patient by delivering a polymer agent into a region of cardiac tissue within the heart of the patient.

In one mode of this aspect, the method includes delivering the polymer agent into the region of cardiac tissue without delivering living material such as cells into the region. In another mode, the polymer agent being delivered into the region is a fibrin glue agent. According to one embodiment of this mode, the delivery of the fibrin glue agent includes forming the fibrin glue in-vivo by mixing a first precursor material and a second precursor material within the patient's body.

Another aspect of the invention is a method for treating a medical condition associated with a heart of a patient by delivering a material that includes collagen or a precursor or analog or derivative thereof into a region of cardiac tissue within the heart of the patient.

Other aspects of the invention include providing various preparations of materials and assembled systems for use in forming non-ablative conduction blocks.

One such aspect includes preparing a cell culture together with a non-living material agent that is adapted to enhance retention of the cells in myocardial tissues, and/or enhance insulative properties of the delivered cellular medium, and/or enhance longevity and viability of the cells once delivered.

One mode of this aspect includes providing the cell culture in combination with a polymer agent, which may be biological or non-biological or a combination of both.

Another mode includes providing the cell culture in combination with a fibrin glue agent.

Another mode includes providing such combination of agents in further combination with a cardiac delivery system that is adapted to provide for percutaneous translumenal delivery of the combined living and non-living agents to the target cardiac location. One beneficial embodiment of such a system may include coupling the living and non-living agents to one delivery catheter adapted to deliver all the component agents to the desired cardiac location.

Another aspect includes providing an overall system that includes: a cardiac conduction mapping system that is adapted to be used to identify the source and/or location of a cardiac arrhythmia; a preparation of non-ablative material agent that is adapted to be injected into a cardiac tissue site and provide non-ablative insulation to cardiac conduction at the location; and a delivery catheter that is adapted to deliver the preparation of non-ablative material agent to the location so as to insulate the location against conducting cardiac signals and thereby reduce or eliminate the arrhythmia.

Another aspect includes: choosing a delivery catheter from a plurality of delivery catheters based upon a known pattern or location where a conduction block is to be performed, wherein the chosen catheter is adapted to deliver a preparation of non-ablative material agent into a cardiac tissue structure at a location within a heart of a patient that is diagnosed as being either a source of arrhythmia or along a arrhythmic pathway; and coupling the delivery catheter with a volume of non-ablative material agent that is adapted to provide substantial insulation against cardiac conduction within the cardiac tissue without ablating the tissue.

A further mode of this aspect includes coupling an injector with the delivery catheter that is adapted to inject the volume of non-ablative material agent to the location via the delivery catheter.

Another mode includes coupling the delivery catheter to a volume of living cells that are adapted to provide at least in part the non-ablative insulation. A further embodiment of this mode includes coupling the delivery catheter to a second non-living material agent that is adapted to enhance retention of the cells, and/or insulation against cardiac conduction, and/or longevity or viability of the cells when delivered into the cardiac tissue structure. A further variation includes providing the second non-living material as a polymer agent. Another variation considered highly beneficial includes providing the non-living material agent as fibrin glue, which may be for example in two-parts.

Another aspect of the invention includes coupling a fibrin glue agent and a volume of living cells to a single delivery catheter that is adapted to deliver the fibrin glue agent and the living cells into a cardiac tissue structure at a location that is either a source of cardiac arrhythmia or along an arrhythmic pathway.

According to one mode of this aspect, the living cells include skeletal myoblasts.

According to another mode, the living cells include stem cells.

According to another mode, the living cells include fibroblasts.

Another aspect of the invention is a system for treating cardiac arrhythmia in a patient that includes a cardiac delivery system and a source of material coupled to the cardiac delivery system. The cardiac delivery system is adapted to deliver a volume of material from the source and substantially along a patterned region of tissue at a location within a tissue structure associated with the patient's heart and that includes cardiac cells. The material is characterized as being substantially non-ablative with respect to cardiac cells, and is adapted to form a substantially non-ablative conduction block along the patterned region of tissue at the location.

According to one mode of this aspect, the cardiac delivery system further includes a contact member that is adapted to substantially contact the patterned region of tissue. The cardiac delivery system is adapted to deliver the material substantially along the patterned region of tissue when the contact member is substantially contacting the region of tissue.

In one embodiment of this mode, the cardiac delivery system is adapted to deliver the volume of material along an elongated pattern of tissue in the region of tissue at the location. In another embodiment, the cardiac delivery system is adapted to deliver the volume of material along a linear pattern of tissue in the region of tissue at the location. In another embodiment, the cardiac delivery system is adapted to deliver the volume of material along a curvilinear pattern of tissue in the region at the location.

In another embodiment of this mode, the cardiac delivery system is adapted to deliver the volume of material substantially along a circumferential region of tissue at the location so as to form a substantially circumferential conduction block at the location. According to one beneficial variation of this embodiment, the cardiac delivery system is adapted to deliver the volume of material along a circumferential region of tissue at the location where a pulmonary vein extends from an atrium. In another variation, a contact member is provided that is adapted to engage the circumferential region of tissue and to deliver the volume of material to the circumferential region of tissue when contacted by the contact member. According to one beneficial feature of this variation, the contact member may be an expandable member, such as an inflatable balloon. Further to this latter variation, the cardiac delivery system may be beneficially adapted to deliver the material to the circumferential region of tissue when the circumferential region of tissue is engaged by the inflatable balloon.

According to another mode, the cardiac delivery system further includes a plurality of needles cooperating with the contact member. The cardiac delivery system is further adapted to deliver the plurality of needles into and substantially along the patterned region of tissue and to inject the material substantially into and along the patterned region of tissue at the location via the needles.

In further modes, the material may be living cells, such as myoblasts or stem cells, or may be a non-living material, such as a biopolymer, and may be a fibrin glue agent that may for example include a first precursor material and a second precursor material. Or, the biopolymer in a further beneficial embodiment may be a collagen agent that may itself be collagen or an analog or derivative thereof.

In still further modes, the source of material may include a first material that comprises living cells, and also a second material that is non-living and that is adapted to enhance formation of the conduction block. In certain embodiments, the second material comprises a polymer agent such as fibrin glue agent or collagen agent. In another regard, the second material may be of a type that is adapted to enhance retention of the living cells at the location, and/or may be of a type of material that is adapted to insulate conduction via gap-junctions between adjacent cells at the location.

Another aspect of the invention is a system for treating a cardiac arrhythmia in a heart of a patient that includes a cardiac delivery system with a contact member and also with a plurality of needles cooperating with the contact member, and a source of material that is adapted to be coupled to the cardiac delivery system. The contact member is adapted to be delivered to the location and to substantially contact a patterned region of tissue at a location associated with the arrhythmia and that includes cardiac cells. The plurality of needles are adapted to be inserted into and substantially along the patterned region of tissue when the contact member is contacted with the patterned region of tissue. The cardiac delivery system is adapted to be coupled to the source of material and to deliver a volume of the material from the source into and substantially along the patterned region of tissue via the plurality of needles. Furthermore, the material is substantially non-ablative to the cardiac tissue and is adapted to form a substantially non-ablative conduction block along the patterned region of tissue at the location.

Another aspect of the invention is a system for treating a cardiac arrhythmia in a patient that includes a cardiac delivery system and a source of material coupled to the cardiac delivery system as follows. The cardiac delivery system includes an expandable member, the source of material includes living cells, and the cardiac delivery system is adapted to deliver the living cells from the source to a region of tissue at a location associated with the cardiac arrhythmia and that includes cardiac cells. In addition, the material is adapted to form a conduction block in the region.

Another aspect of the invention is a system for treating a cardiac arrhythmia in a patient that includes a cardiac delivery device with a guidewire tracking member, a guidewire adapted to slideably engage the guidewire tracking member, and a source of material coupled to the cardiac delivery device, and the system is further characterized as follows. The cardiac delivery device is adapted to track over the guidewire to a location associated with the cardiac arrhythmia, and is further adapted to deliver a volume of the material from the source into a region of tissue at the location and that includes cardiac cells. The material is characterized as being substantially non-ablative to cardiac cells, but is adapted to form a conduction block in the region.

Another aspect of the invention is a system for treating a cardiac arrhythmia in a patient that includes a cardiac delivery device and a source of material coupled to the cardiac delivery device, and the system is further characterized as follows. The cardiac delivery device is adapted to deliver a volume of the material from the source and to a circumferential region of tissue at a location where a pulmonary vein extends from an atrium while the cardiac delivery device is substantially secured at a position at the location. The cardiac delivery device is further adapted to allow blood to perfuse downstream across the location while delivering the material into the circumferential region of tissue. The material is characterized as being substantially non-ablative to cardiac tissue, but is adapted to form a conduction block in the circumferential region of tissue.

It is to be appreciated that various further modes are contemplated using fibroblast cells according to the various cellular therapy aspects of the invention elsewhere herein described, or may be considered further embodiments of the various modes of those inventive aspects, or to be variations of elsewhere described embodiments of such modes, as considered appropriate according to one of ordinary skill.

For example, one such further mode includes introducing autologous fibroblasts into a region of a patient's heart as an insulator to thereby create a conduction block sufficient to treat cardiac arrhythmias.

According to one embodiment of such mode, the fibroblasts are autologous. According to one variation of this embodiment, the autologous fibroblasts are derived from a biopsy of a patient's skin, amplified, and injected and/or grafted. In one further variation of this embodiment, such fibroblasts are removed from the patient and prepared in a manner that is adapted to be delivered to the desired region of the heart. A further feature of this variation includes coupling such preparation to an appropriate delivery catheter.

According to another embodiment, the fibroblasts are delivered in a manner adapted to electrically isolate one or more arrhythmogenic foci in a patient's pulmonary vein.

According to another embodiment, the fibroblasts are delivered in a manner adapted to treat atrial fibrillation.

According to another embodiment, the autologous fibroblasts are delivered into a location associated with a patient's pulmonary vein to create an encircling isolated region from the mitral annulus to insulate and reduce and/or block electrical/mechanical conduction between the pulmonary vein and the atrium and/or atrial appendage.

According to one highly beneficial variation of this embodiment, the fibroblasts are delivered into and substantially along a circumferential region of tissue at a location where the pulmonary vein extends from the atrium, which location may be for example where at the pulmonary vein ostium which may be a funneling region where the atrium transitions into the pulmonary vein, or along a region where cardiac tissue extends into the pulmonary vein, or along the atrial wall and immediately surrounding the pulmonary vein ostium.

Another embodiment includes placing autologous fibroblasts into a patient's pulmonary vein to disrupt the electrical conduction between the atria and/or atrial appendage and the pulmonary vein to restore sinus rhythm and reduce, eliminate, or prevent the incidence of atrial fibrillation.

Accordingly, this embodiment according to one beneficial variation includes coupling a preparation of such fibroblasts for delivery with a pulmonary vein delivery catheter that is adapted to deliver the fibroblasts to produce the results described.

Another embodiment of this fibroblast therapy method includes introducing the autologous fibroblasts into a patient's pulmonary vein to disrupt the electrical conduction between the atria and the pulmonary vein to reduce, eliminate, or prevent atrial fibrillation.

Another object of certain of the fibroblast modes and embodiments of the invention is to provide a method of introducing autologous fibroblasts in place of ablative therapy, e.g. microwave, thermal, RF, ultrasound, or laser energy delivery modalities, or chemical ablation such as alcohol ablation, in order to isolate a patient's pulmonary vein from the atria and/or atrial appendage and restore sinus rhythm and/or reduce or eliminate the occurrence of atrial fibrillation.

Another embodiment of the fibroblast therapy method includes introducing modified autologous fibroblasts into arrhythmogenic foci as insulators to electrically isolate arrhythmogenic foci for the treatment of atrial fibrillation.

Another embodiment of this fibroblast therapy mode includes introducing modified autologous fibroblasts into a patient's pulmonary vein to create an encircling isolated region from the mitral annulus to insulate and reduce and/or block electrical/mechanical conduction between the pulmonary vein and the atrium and/or atrial appendage. In one further variation of this embodiment, the modified autologous fibroblasts are injected.

Another embodiment of the fibroblast therapy mode includes introducing modified autologous fibroblasts into a patient's pulmonary vein to disrupt the electrical conduction between the atria and/or atrial appendage and the pulmonary vein to substantially restore sinus rhythm, or at least reduce the incidence of atrial fibrillation. In one beneficial variation of this embodiment, the autologous fibroblasts may be derived from a biopsy of the patient's skin, amplified, and injected, and/or grafted.

Still a further fibroblast therapy embodiment includes introducing modified autologous fibroblasts into a patient's pulmonary vein to disrupt the electrical conduction between the atria and the pulmonary vein to reduce or eliminate atrial fibrillation. In a highly beneficial variation, the autologous fibroblasts are derived from a biopsy of the patient's heart, amplified, and injected, and/or grafted.

Another object of certain of the fibroblast therapy modes of the invention is to provide a method that introduces autologous fibroblasts in place of microwave, thermal, RF, ultrasound, or laser energy to isolate a patient's pulmonary vein from the atria and and/or atrial appendage and restore sinus rhythm and/or reduce or eliminate the occurrence of atrial fibrillation.

Another fibroblast embodiment includes a method of delivering autologous fibroblasts into the arrhythmogenic foci to electrically isolate the foci to reduce or eliminate the arrhythmogenic conduction pathway producing ventricular or atrial fibrillation or tachyarrhythmia, using a catheter and needle injection system so that the fibroblasts can be delivered percutaneously.

Further aspects, modes, embodiments, variations, and features of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1 is a schematic view of various components of a system for creating cardiac conduction blocks according to one embodiment of the invention.

FIG. 2A is a transverse cross-sectional view of one catheter embodiment such as taken along line 2-2 through the catheter shown in the system of FIG. 1.

FIG. 2B is a transverse cross-sectional view according to another catheter embodiment in a similar view to that shown in FIG. 2A.

FIG. 2C is a transverse cross-sectional view according to still another catheter embodiment in a similar view to that shown in FIG. 2A.

FIG. 3 is a schematic view of various components of another system for creating cardiac conduction blocks according to another embodiment of the invention.

FIG. 4 is an exploded view of a distal tip portion of a needle according to one further embodiment for use according to a system of the invention such as that shown in FIG. 3.

FIG. 5 shows an exploded view of a drop of material agent delivered through a needle according to the invention as shown in region 5 in FIG. 3.

FIG. 6 shows a partially cross-sectioned view of a distal tip portion of another non-ablative material delivery system for forming a cardiac conduction block according to another embodiment of the invention.

FIGS. 7A-C show exploded views of an infarct region of a cardiac chamber during sequential modes of using the present invention, respectively.

FIG. 8A shows a partially segmented perspective view of a distal end portion of another system according to a further embodiment of the invention.

FIG. 8B shows an end view taken along lines B-B in FIG. 8A.

FIG. 9 shows a partially segmented view of a distal end portion of the device shown in FIGS. 9A-B during one mode of in-vivo use at a location where a pulmonary vein extends from an atrium in a patient.

FIG. 10 shows a schematic view of another catheter embodiment according to the invention.

FIG. 11 shows a schematic view of yet another catheter embodiment of the invention.

FIGS. 12A-D show various modes of forming a patterned conduction block for pulmonary vein isolation according to certain embodiments of the invention.

FIGS. 13A-B show various modes of another embodiment of the invention for forming a patterned conduction block for pulmonary vein isolation.

FIGS. 14A-C show various further modes providing elongated patterned conduction blocks according to the invention.

FIG. 15 shows various steps in forming a system, for delivering cells in combination with fibrin glue to form a conduction block according to a further embodiment of the invention.

FIGS. 16A-B show schematic view of two representative cardiac cells during two modes according to the invention, wherein FIG. 16B shows the cells physically separated by injection of a material into the junction between the cells according to one embodiment of the invention.

### DETAILED DESCRIPTION OF THE INVENTION

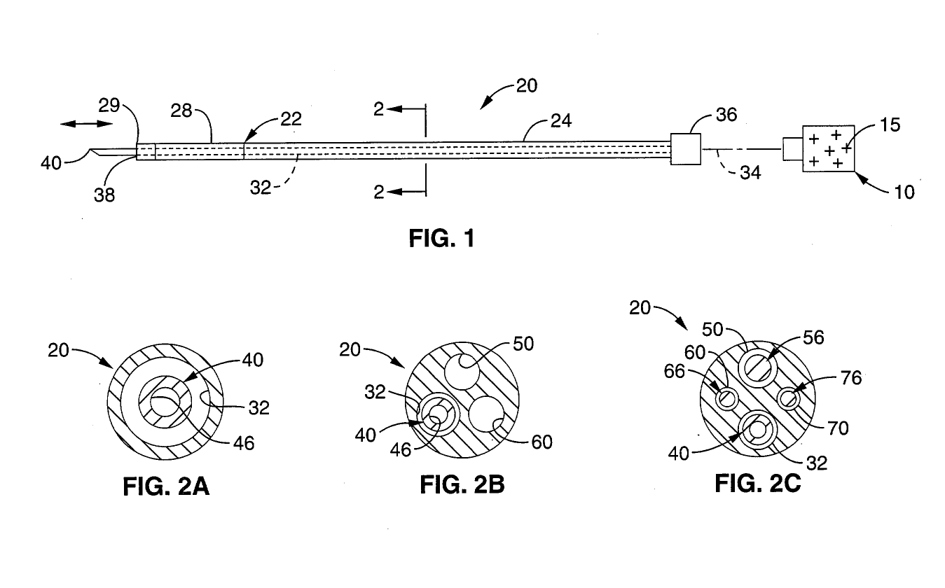

Referring more specifically to the drawings, for illustrative purposes the present invention is embodied in the systems and methods generally shown in FIG. 1 through FIG. 16B. It will be appreciated that the apparatus may vary as to configuration and as to details of the parts, and that the method may vary as to the specific steps and sequence, without departing from the basic concepts as disclosed herein.

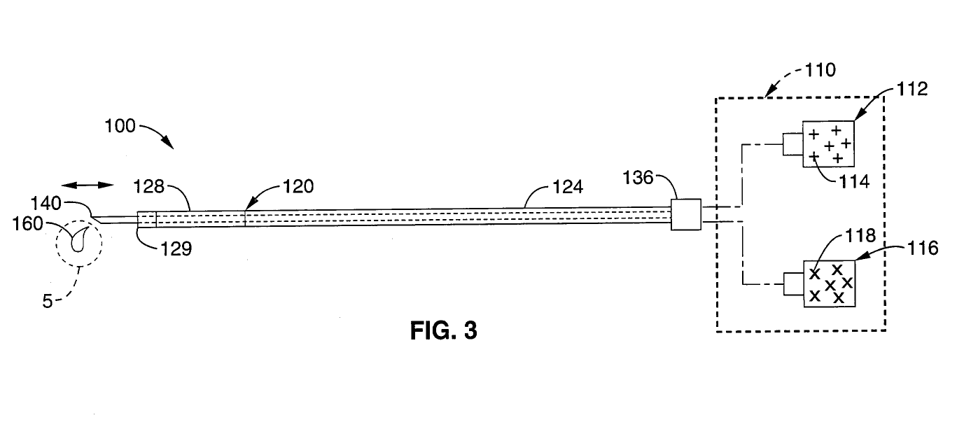

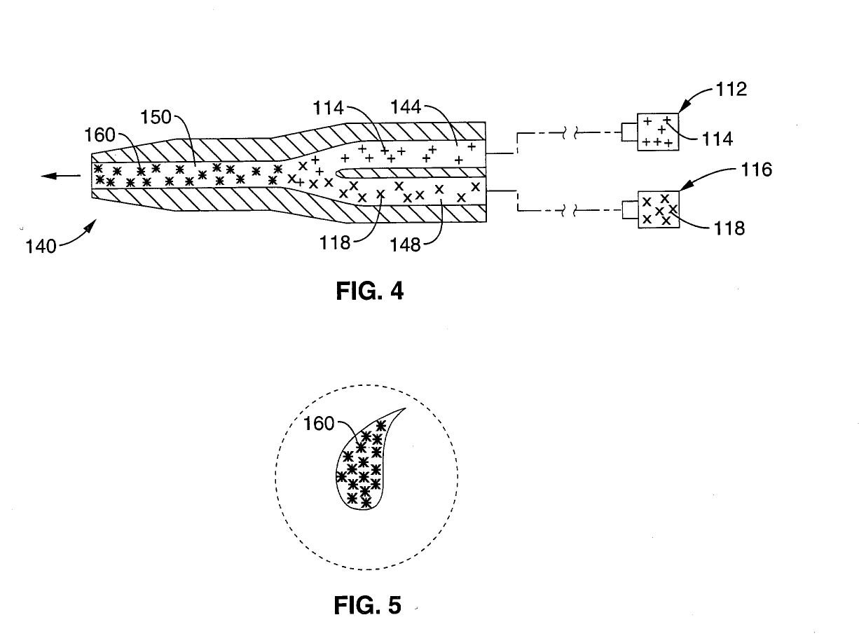

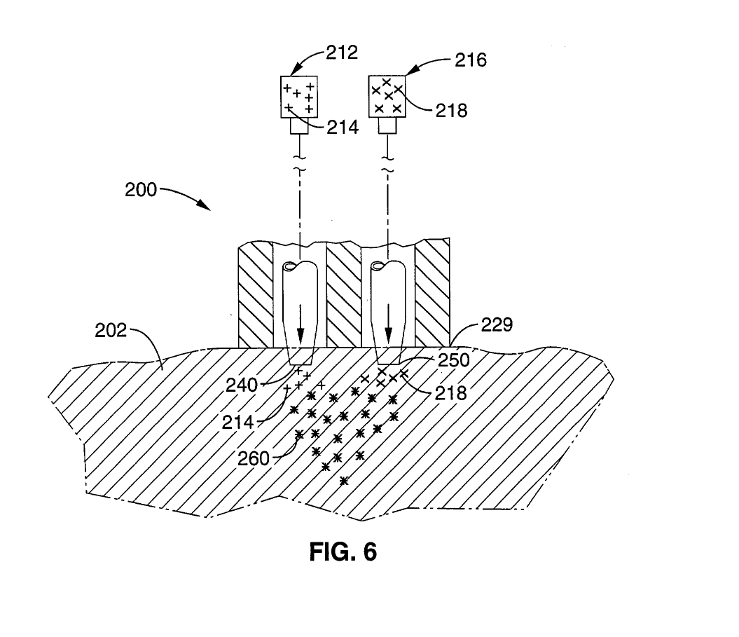

FIG. 1 shows one embodiment of the invention that provides a cardiac treatment system 1 that includes a source of material 10 and a delivery catheter 20. Delivery catheter 20 is adapted to couple to source of material 10 and to deliver material 15 to a region of a heart in a patient, as shown for example in FIG. 2. More specifically, according to this embodiment, delivery catheter 20 has an elongate body 22 with a proximal end portion 24, a distal end portion 28, and a lumen 32 extending therethrough between proximal and distal ports 34,38 located along proximal and distal end portions 24,26, respectively. Proximal port 34 includes a proximal coupler 36 that is adapted to couple to a coupler (not shown) on source of material 10.

Delivery catheter 20 includes a needle 40 that is adapted to extend beyond distal tip 29 of catheter 20 and into tissue and further to deliver material 15 from source 10 into such tissue. Needle 40 may be fixed relative to catheter 20, or in a beneficial variation is moveable, such as axially, as shown in FIG. 1 by axial reference arrow.

The assembly of delivery catheter 20 and needle 40, in a highly simplified form, may include simply a single lumen shaft for catheter body 20 having a single lumen 32 which slideably houses needle 40 that further includes its own delivery lumen 46 for delivering material 15 as an agent into the target tissue. This arrangement is shown for example in cross-section in FIG. 2A. Alternatively, a multi-lumen design may be incorporated, as shown in variations in FIGS. 2B-C as follows.

FIG. 2B shows a cross section of a multi-lumen design with needle 40 residing within catheter lumen 32, and also further providing additional lumens 50 and 60 in catheter 20. These additional lumens may have various different functions, depending upon the particular needs.

In the particular variation shown in FIG. 2C, lumen 50 houses a pull-wire 56, whereas lumens 60 and 70 house lead wires 66 and 76. Pull-wire 56 extends between a first securement point at tip 29 and an actuator (not shown) along proximal end portion 24 that is adapted to allow for axial manipulation of pull-wire externally of the body, to thereby deflect distal end portion 28 in-vivo. For deflectable tip designs, certain other material properties are generally taken into account, such as catheter shaft design, flexibility of material chosen for shaft construction, etc., and various other substitute deflection or other manipulation designs or techniques are also contemplated. For example, rather than pull-wire, push wires may be used, or other members than wires such as polymer filaments or fibers, or torsional members. In another alternative design not shown, a guidewire tracking member is provided to work over a guidewire as a rail for remote positioning in-vivo.

Lead wires 66 and 76 extend between a mapping electrode, such as may be provided at tip 29 or otherwise along distal end portion 28, and a proximal electrical coupler that is adapted to couple to a mapping monitoring assembly to provide an overall mapping system with catheter 20 for determining the location for material injection to form a conduction block. General mapping electrode configurations, or combinations of such electrodes, may be suitable for such use according to one of ordinary skill. Moreover, the mapping electrode may be radiopaque for x-ray visualization. To this end, other radiopaque tip markers may also be deployed for such visualization, or other markers or visualization techniques may be used according to one of ordinary skill, such as ultrasound (for example either intravascular, intracardiac, or transesophageal), magnetic resonance imaging (“MRI”), or other suitable modes.

It is also contemplated that needle 40 may take many different forms, such as a relatively straight sharp-tip needle, or may be a hollow screw-shaped needle or other mechanism, such as to aid in anchoring at the desired location.

Moreover, catheter 20 may be adapted to provide delivery of needle 40 at other places than at tip 29, such as along the side wall of the elongate body of distal end portion 28 of catheter. In addition, multiple needles may be deployed such as along a length of catheter 20 in order to form conduction blocks along a prescribed length. To that end, the same needle may be used at different locations, such as delivery through different lumens to different ports along catheter 20, or multiple needles deployed simultaneously or sequentially.

Source of material 10 includes an injectable material 15 that is adapted to form a conduction block in cardiac tissue structures without substantially ablating the cardiac tissue. Examples of highly beneficial materials for use according to the invention include: cells, polymers, or other fluids or preparations that interfere with intercellular junctions, such as impeding communication across or physically separating cellular gap junctions. Another highly beneficial example includes an injectable material containing collagen, or a precursor or analog or derivative thereof.

More specific modes of the invention using cells include myoblasts, fibroblasts, stem cells, or other suitable cells that provide sufficient gap junctions with cardiac cells to form the desired conduction block. With further respect to cell delivery, they may be cultured from the patient's own cells, or may be foreign to the body, such as from a regulated cell culture.

Tissue engineering techniques utilizing skeletal myoblast transplantation for myocardial repair has gained increased attention with the demonstration that skeletal myoblasts survive and form contractile myofibers in normal and injured myocardium. However, the emphasis of myocardial repair has focused on the preservation of myocardial contractility with little attention given to the effects of tissue engineering on cardiac conduction or arrhythmogenesis.

According to embodiments of the present invention using “myoblasts” as a chosen living cell material to be delivered to effect a conduction block, such cells have in the past been observed to create arrhythmias when implanted into normal cardiac tissue structures, which observation is believed to result from blocking normal conduction pathways due to gap junction deficiencies between the transplanted cells and existing cardiac tissue. This has been viewed as a problem due to the prior attempts at increasing contractility and conduction with the cell therapy. In contrast, use of myoblast transplantation according to the present invention adapts delivery of these cells in a highly localized manner at locations along arrhythmic pathways, or at focal sources of arrhythmia, in order to focus the conduction blocking effects in a positive manner to in fact provide the opposite results versus previous observations—cure arrhythmias with localized, cellular conduction blocks.

Fibroblasts are another alternative cell of the type considered highly beneficial mode for creating conduction blocks via cell therapy. In one particular beneficial regard, fibroblasts do not undergo a transition stage from proliferating to mature cells such as skeletal myoblasts. Fibroblasts therefore have a more homogeneous excitation pattern as compared to skeletal muscle. Fibroblasts' electrophysiological properties are fairly consistent from one fibroblast to the next, and are believed to be effective for blocking conduction. Therefore, in one illustrative embodiment using fibroblasts to block VT for example, very similar responses can be predicted between batches/injections.

Therefore the invention according to a further embodiment provides systems and methods to treat cardiac conduction disturbances using fibroblast cell transplantation. More specifically, fibroblasts according to a highly beneficial variation of such embodiment are taken from dermal samples, and are subsequently prepared appropriately and transplanted to a location within a cardiac tissue structure to facilitate cardiac tissue conduction or creation of alternate pathways of conduction to treat conduction disturbances in the heart, such as atrial fibrillation, ventricular tachycardia and/or ventricular arrhythmias and CHF.

The invention therefore according to this beneficial embodiment uses fibroblasts from the patient's own body, and transplanting them to the area of the conduction abnormality of the heart. Fibroblasts are cells that can survive and multiply in the low oxygen environment of the scar (typically conduction abnormalities of the heart occur on the leading edge between the scar tissue from an AMI and normal cardiac tissue) and have the ability to either block or change/remodel the conduction pathway of the heart or where electromechanical coupling of the fibroblasts can be induced, create new pathways to normalize the conduction of the heart from abnormal conduction pathways.

Yair Feld, et. al., “Electrophysiological Modulation of Cardiomyocytic Tissue by Transfected Fibroblasts Expressing Potassium Channels: A Novel Strategy to Manipulate Exitability,” Circulation, Jan. 29, 2002 pgs 522-529, disclosed that fibroblasts transfected with voltage-sensitive potassium channel Kv 1.3 may modify the electrophysiological properties of a cardiomyocyte culture. They disclosed in vitro that fibroblasts may be able to electrically couple with cardiac myocytes to cause changes in electrophysiological properties. The disclosure of this reference is herein incorporated in its entirety by reference thereto.

Therefore, according to certain particular embodiments of the present invention, a patient's own fibroblasts are cultured and transplanted into identified areas of conduction abnormalities in the heart where they can proliferate and act as a blocking agent to remodel the conduction pathway. Or, methods may be employed to include the production of gap junction proteins in these fibroblast cells in order to utilize them via transplantation into scarred areas of the heart to normalize the conduction pathway via the fibroblasts' ability to electromechanically couple with the existing cardiac myoctyes.

Whereas certain broad aspects of the invention incorporate cell therapy in general for creating conduction blocks to treat arrhythmias, certain more specific modes are considered also independently beneficial. For example, in one particular such mode autologous fibroblasts are used for the treatment of AF. Fibroblasts are a cell line that typically is associated with tissue damage (i.e., skin damage, AMI) and healing of tissue to produce scar. Activation of fibroblasts occurs in response to injury. These events cause a transition of cell types to activated phenotypes having fundamentally different biologic function from corresponding quiescent cells in normal tissue. These cellular phenotypes (arising from coordinated gene expression) are regulated by cytokines, growth factors, and down stream nuclear targets. As in the example of wound healing, fibroblasts are directed to the repair and rebuilding of tissue. Quiescent fibroblasts in normal tissue primarily are responsible for steady-state turnover of extracellular matrix, as disclosed for example in the following references: EGHBALI M, CZAJA M J, ZEYDEL M, et al., “Collagen chain mRNAs in isolated heart cells from young adult rats,” J Mol Cell Biol 1988; 20: 267-276; and POSTLETHWAITE A, KANG A., “Fibroblasts and matrix proteins; and Gallin J, Snyderman R (eds), “Inflammation. Basic Principles and Clinical Correlates,” 1999, Philadelphia: Lippincott Williams & Wilkins. The disclosures of these references are herein incorporated in their entirety by reference thereto.

Skin fibroblasts potentiate the migration to PDGF and increase collagen accumulation and MMP synthesis, and net collagen accumulation, as disclosed for example in the following reference which is also herein incorporated in its entirety by reference thereto: KAWAGUCHIY, HARA M, WRIGHT T M., “Endogenous 1 alpha from systemic sclerosis fibroblasts induces IL-6 and PDGF-A,” J Clin Invest, 1999, 103:1253-1260. This formation of collagen matrix coupled with the lack of gap junction proteins in fibroblasts creates the electromechanical isolation from cardiomyocytes. In one more particular example, a lack of electrical conduction has been observed in regions of fibroblast migration in the myocardium of patients having a previous MI.

Therefore, fibroblasts are cells that can be utilized (and proliferated) to create electrical insulation and/or reduction of electrical conduction in regions in the myocardium that present as the arrhythmogenic foci of abnormal conduction pathways.

Fibroblasts can be biopsied from many tissues in the body (lungs, heart, skin) isolated, amplified in culture, and introduced (via injection, graft delivery, grafting, with a polymetric carrier or backbone) into a region of the heart where there is a need to reduce the conduction, isolate an arrhythmic pathway, or isolate an arrhythmogenic focus in the cardiovascular system including pulmonary veins, atria and ventricles, and atrial appendage.

Cell therapy for treating cardiac arrhythmias according to various of the present embodiments is considered one mode (though highly beneficial) of a still broader aspect of the invention which provides a non-ablative means for creating conduction blocks in cardiac tissue structures, more specifically associated with the cardiac chambers. This aspect provides immense benefit in providing the intended therapy without many of the other side effects and shortcomings of other conventional techniques for forming cardiac conduction blocks, such as in particular using cardiac ablation.

For example, hyperthermia and thus collagen shrinkage and other substantial scarring responses to other conventional ablation energy delivery modalities is substantially avoided. This has particular benefit for example in preventing occlusion, such as in forming conduction blocks in or around a location where a pulmonary vein extends from an atrium in order to treat or prevent atrial fibrillation.

In addition, cell therapy is generally accomplished in a highly localized manner, whereas many ablation techniques suffer from control of energy delivery and extent of impact therefrom in tissues at or beyond the targeted location. For example, charring associated with the high temperature gradient necessary to form transmural conduction blocks using many RF energy ablation devices techniques is avoided. In another regard, undesired energy dissipation into surrounding tissues is often observed using many conventional ablation techniques and is also avoided using the substantially non-ablative cellular therapy systems and methods of the present invention.

Accordingly, the present invention contemplates a broad scope with respect to providing conduction blocks to treat cardiac arrhythmias without substantially ablating cardiac tissue. As such, other suitable modes than cellular therapy are contemplated according to this aspect of the invention.

For example, a further highly beneficial embodiment of the invention provides a system and method for delivering a non-ablative, non-living media into a region of cardiac tissue for the purpose of forming a cardiac conduction block there. More specifically, certain biopolymer agents such as fibrin glue agent may be highly beneficial agents for such delivery and use.

In another example, collagen, or precursor or analog or derivative materials thereof, is further considered a highly beneficial agent for this purpose, in particular in injectable form, which may further include for example a carrier or matrix that adapts the collagen for delivery and may or may not be otherwise be retained with the collagen when implanted to the location, or may otherwise be transported or metabolized, etc., at the injection site.

Embodiments of material 15 may include primarily or only one material such as according to the examples above, or may include combinations of materials. For example, embodiments of material 15 that includes cells may include other materials, such as fluids or other substrates to provide the cells in an overall preparation as a cellular media that is adapted to be injected, such as in particular through delivery lumen 32 of delivery catheter 20. In one particular example that has been observed to be useful, material 15 may include skeletal myoblasts or other suitable substitute cells in combination with a biopolymer agent such as fibrin glue agent, which may itself be provided as two precursor materials that are mixed to form fibrin glue that assists in forming the conduction block when delivered with cells at the desired location within the heart. Collagen or preparations thereof, including precursors or analogs or derivatives of collagen, is also considered useful in such combination.

In general, a “polymer” is herein defined as a chain of multiple units or “mers”. Fibrin glue for example contains polymerized fibrin monomers, and is further herein considered an illustrative example of a biopolymer since its components are biological. Thrombin in a kit is an initiator or catalyst which enzymatically cleaves fibrinogen into fibrin. The monomers can then polymerize into a fibrin gel or glue. Further more detailed examples of fibrin glues that may be useful according to various aspects of the present invention are disclosed in the following reference: Sierra, DH, “Fibrin sealant adhesive systems: a review of their chemistry, material properties and clinical applications.” J Biomater Appl. 1993;7:309-52. The disclosure of this reference is herein incorporated in its entirety by reference thereto.

According to still a further embodiment of the invention, a preparation of living material, such as for example cells, in combination with a non-living material is delivered into cardiac tissue structures to form a conduction block there. In one further more detailed embodiment, the non-living material is adapted to enhance retention of the cells being delivered into the location where the conduction block is to be formed. In another regard, the non-living material is adapted to further contribute to forming the conduction block, such as by intervening to the gap-junctions between cells in the injected region. One particular example of a material that provides significant benefit in such combination with cellular therapy is fibrin glue. More specifically, fibrin glue has been observed to provide enhanced retention of cells such as myoblasts that are injected into cardiac tissue in order to treat damaged cardiac structures, such as infarct regions of a heart, as further developed by reference to one of the Examples below.

Notwithstanding the significant benefit of using fibrin glue in combination with cell delivery for treating cardiac arrhythmias, other suitable substitute materials having similarly beneficial effects in such combination are also contemplated, such as other polymers or molecular scaffolds or materials that intervene sufficiently to inter-cellular gap junctions or otherwise impact the extracellular matrix in cardiac tissue structures to substantially block arrhythmic conduction from propagating. Moreover, collagen or precursors or analogs or derivatives thereof are further considered useful for this purpose, either in addition or in the alternative to fibrin glue.

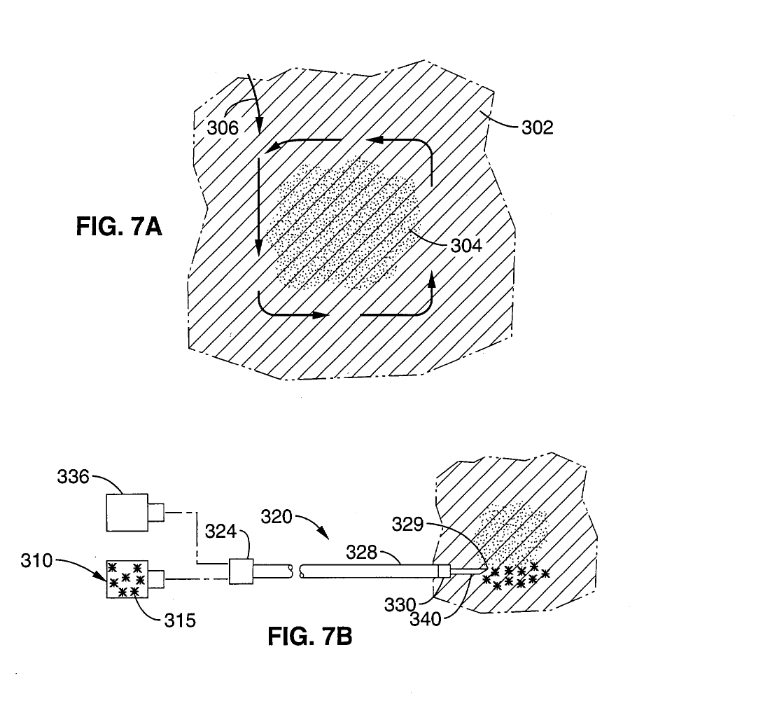

For further illustration, FIG. 3 shows a further embodiment of the invention that provides a delivery catheter 120 that is adapted to couple to two sources 112,116 of two separate materials 114,118, respectively. In this regard, such combination is considered where reference to a “source of material” is elsewhere herein described, and is thus illustrated as a combination source of material 110 in FIG. 3. In this particular embodiment, the two materials 114,118 are two precursor materials to forming fibrin glue, and their combined delivery, either as the separate precursor materials that are later mixed, or in combined form mixed as fibrin glue, is hence considered a fibrin glue “agent”. Thus, “agent” in this use is intended to mean the end result, or the necessary combination of precursor material components that lead to the resultant material, though in other regards “agent” may also include the desired resulting material itself.

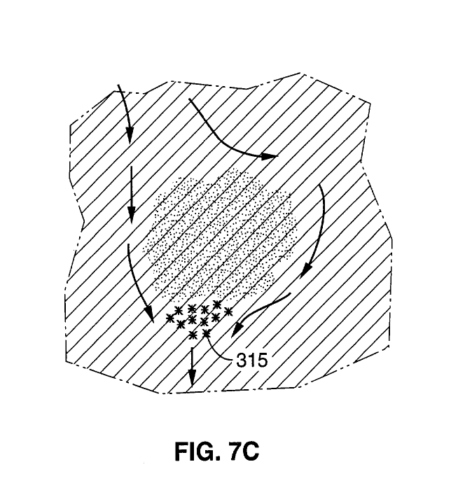

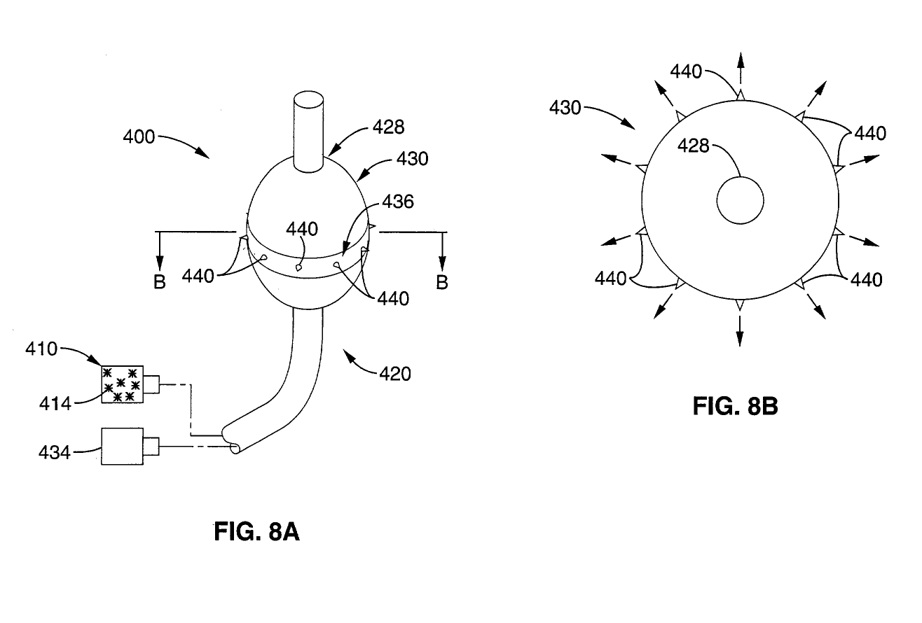

Accordingly, a system 100 as shown in FIG. 3 and by further reference to FIGS. 4 and 5, is adapted to deliver precursor materials 114,118 into the body separately, where they are therein mixed and delivered through needle 140 beyond tip 129 of distal end portion 128 into tissue as a mixed form of fibrin glue 160. An exemplary needle assembly 140 shown in FIG. 5 for accomplishing this objective delivers precursor materials 114,118 via separate lumens 144,148, respectively, that converge into mixing lumen 150 related to needle assembly 140 wherein fibrin glue 160 is formed just prior to injection via needle 140 as an injected fibrin glue, as shown in exploded view in FIG. 5.

It is contemplated that the assembly and various components of system 100 shown by way of the embodiments in FIGS. 3-5 are illustrative, and other suitable substitutes may be used in order to achieve the objective of delivering two precursor materials and mixing them to form the media for injection. For example, in certain circumstances, they may be mixed prior to delivery into the distal portions of catheter 120, such as at a mixing chamber in proximal coupler 136, or prior to coupling to delivery catheter 120. To this end, one coupler may be used to couple to each of multiple sources of material for delivery, or multiple proximal couplers may be used.

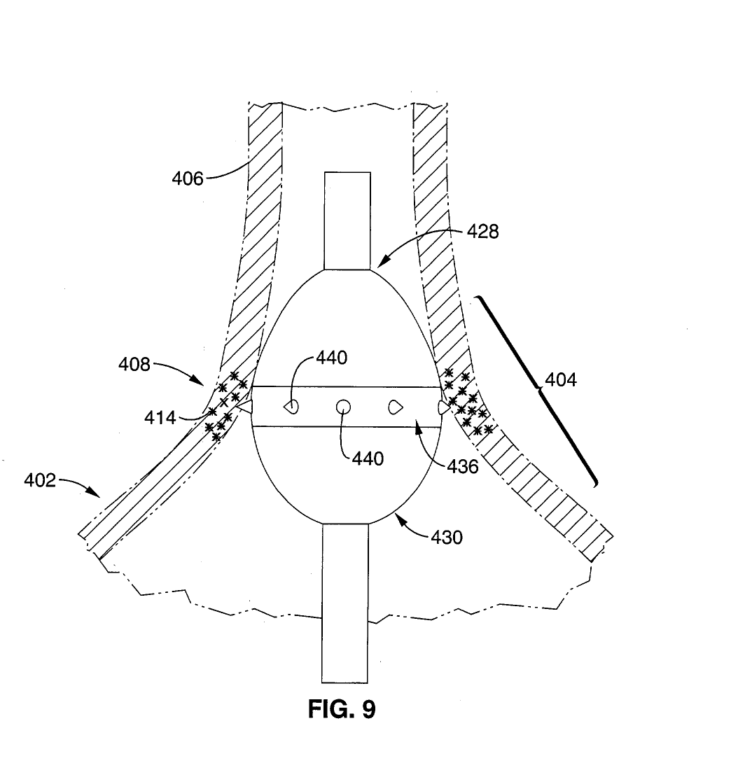

Still further, more than one delivery device may be used for each of two materials being delivered. For example, FIG. 6 shows a schematic view of a system 200 wherein a distal end 229 of catheter 220 in contact with a reference region of cardiac tissue 202. In this embodiment, two separate and distinct needles 240,250 are used to deliver each of two materials 214,218, respectively, from sources 212,216, also respectively, located outside of the patient's body. In this manner, two precursor materials are delivered separately into the tissue 202 where they mix to form fibrin glue 260 within the tissue structure. This provides the benefit of preventing unwanted clogging of the respective delivery lumen within catheter 220 during delivery to the remote in-vivo tissue location. Further to this example, various other structures are assumed to form a part of the overall system 200, such as for catheter 220, including for example an actuator (not shown) that may be one common actuator or multiple independent actuators for advancing needles 240,250 into tissue 202, and/or otherwise injecting the materials 214,218 respectively therethrough.

In addition, the systems 100 and 200 just described are illustrated for use with fibrin glue agents that include a combination of two precursor materials. However, other materials may be substituted for use in such systems, and such systems may be appropriately modified for a particular material delivery. For example, cells may be delivered in combination with a second material according to either system 100 or 200. In addition, such second material may itself be a fibrin glue or other biopolymer agent, which may illustrate further multiples of sources and delivery lumens.

For further understanding, the embodiment of FIGS. 3-4 may be combined with that of FIG. 6 as follows. A source such as source 212 in FIG. 6 may include cells as material 214 to be delivered. However, source 216 in that embodiment may itself include two separate sources that are precursor fibrin glue agent materials, and thus needle 250 of the FIG. 6 embodiment may be of the type shown for needle 140 in FIG. 4.

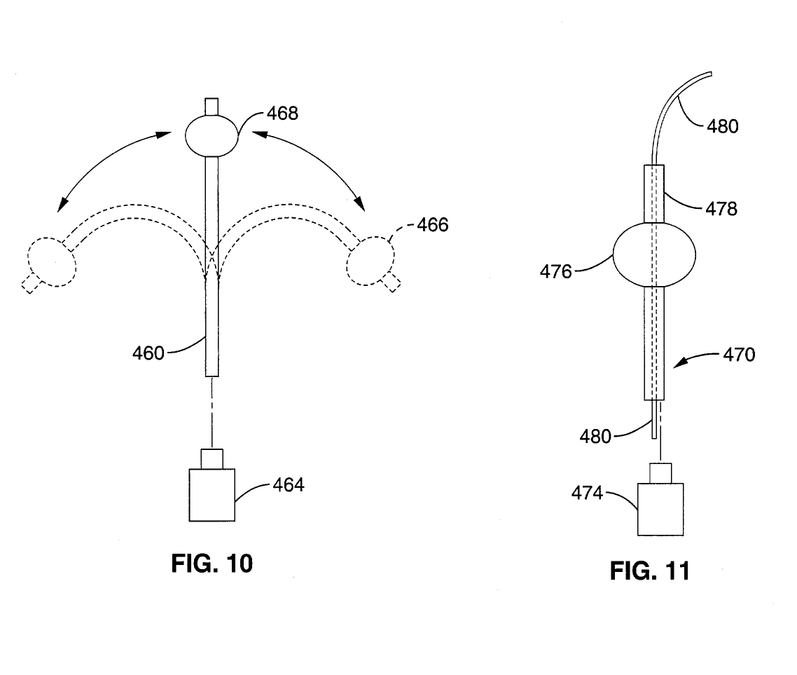

The present invention is useful for treating cardiac arrhythmias, such as for example as follows by reference to FIGS. 7A-C. More specifically, FIG. 7A shows a region of cardiac tissue 302 that includes an infarct zone 304 that is related to a reentrant conduction pathway 306 (illustrated in bolded arrows) associated with cardiac arrhythmia. As shown in FIG. 7B, the distal end portion 328 of a catheter 320 of the invention is delivered to the region at a location associated with the reentrant circuit 306. This is done for example using a mapping electrode 330 provided at distal tip 329 and via an external mapping/monitoring system 336 coupled to proximal end portion 324 of catheter 320 outside of the body. Needle 340 is punctured into the tissue at the location, and is used to inject non-ablative conduction block material 315 from source 310, also coupled to proximal end portion 324 of catheter 320 outside of the body. According to this highly localized injection of the material 315 into the location across the reentrant circuit 306, the circuit is blocked by material 315 and its arrhythmic effects diminished or entirely remedied with hopeful return to sinus rhythm.

Each type of cardiac arrhythmia is also considered to present unique circumstances, both anatomically and functionally, that may in some circumstances benefit from specially adapted cell delivery devices and techniques in order to provide the most appropriate respective anti-arrhythmia therapy. For example, certain arrhythmias require precisely placed conduction blocks to intervene and block their abnormal conduction. Such circumstances may benefit from specially adapted delivery devices and other considerations such as quantity of cells being delivered.

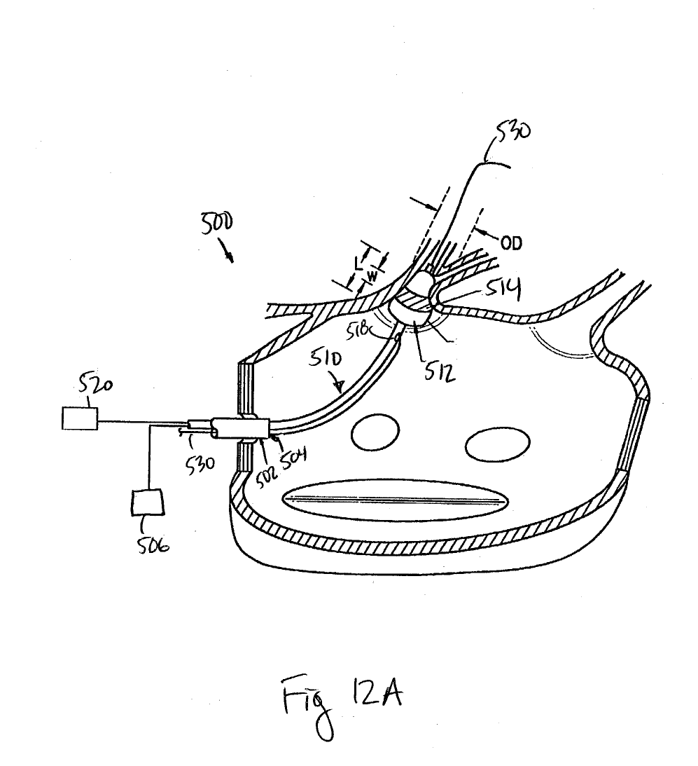

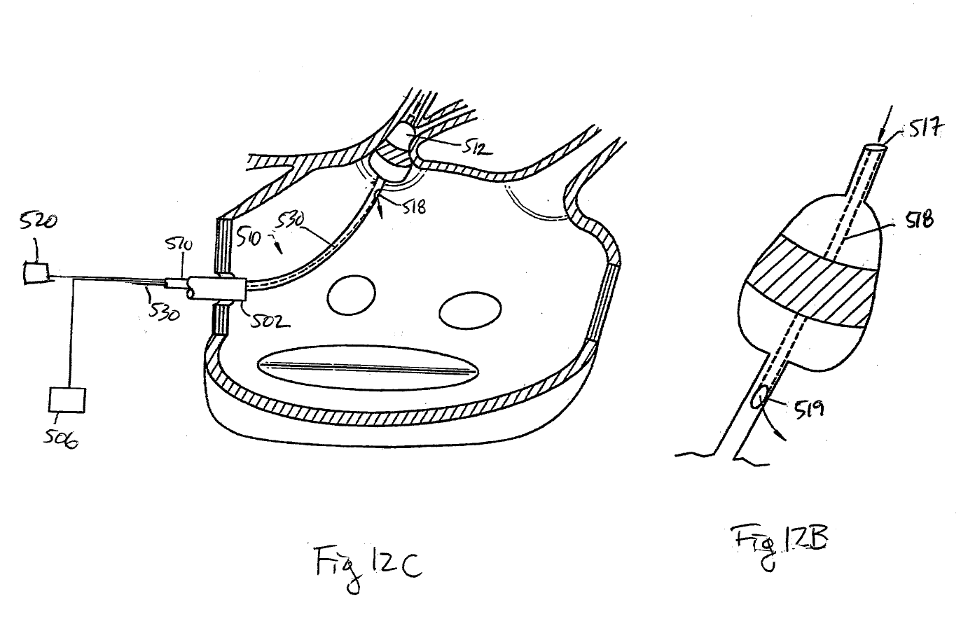

One illustrative example of a highly beneficial embodiment illustrating such a particular adaptation provides a circumferential pattern for delivery of non-ablative conduction block material, and is variously described by reference to the embodiments shown in FIGS. 8A-11 as follows.

System 400 shown in FIG. 8A includes a delivery catheter 420 with an expandable member 430 on its distal end portion 428 and coupled to a proximal actuator 434 externally of the body. More specifically, in the embodiment shown expandable member 430 is an inflatable balloon that is coupled via catheter 420 to actuator 434 that is a source of pressurized fluid. A plurality of needles 440 are provided along a circumferential band 436 of balloon 430, as shown in FIG. 8A and also FIG. 8B.

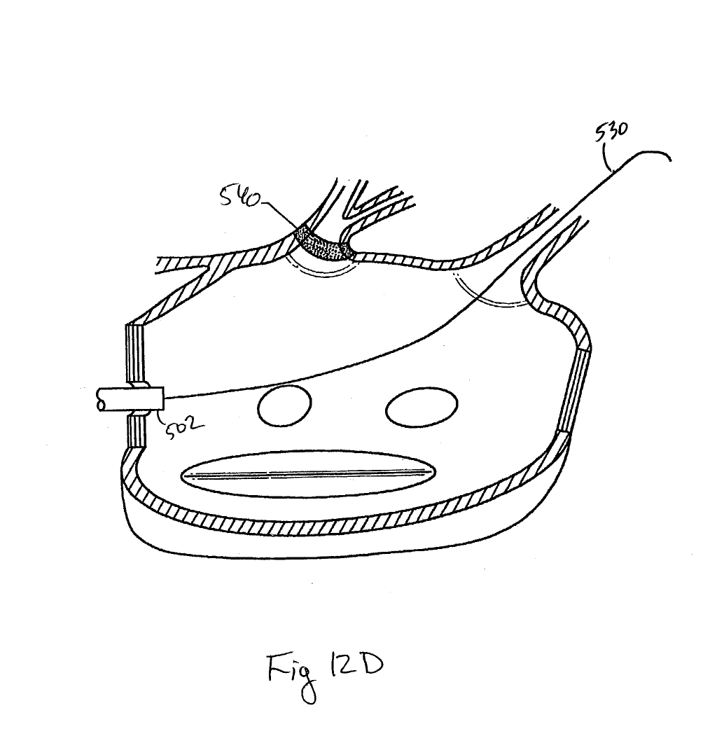

System 400 is in particular adapted for forming non-ablative circumferential conduction block to treat atrial arrhythmia, and still more specifically to form a circumferential conduction block in a circumferential region of tissue at a location where a pulmonary vein extends from an atrium. As shown in FIG. 9, such location may be generally at a funneling region or ostium 404 between the atrium 402 and respective pulmonary vein 406, but may be located up along the pulmonary vein wall itself to the extent cardiac tissue is located there, and is also considered to include a region of tissue along the back wall of the atrium and closely surrounding the pulmonary vein ostium. All of these regions together may be included in a treatment and be considered at a “location where a pulmonary vein extends from an atrium,” or such treatment may be more localized to only one such place, in which case it is still considered a “location where a pulmonary vein extends from an atrium.”

In any event, such circumferential conduction block is adapted to substantially isolate cardiac conduction between tissue located on one side of the circumferential region of tissue, e.g. within the circumference, and tissue on the other side, such as outside of circumferential block. For further illustration, in the highly beneficial mode shown in FIG. 9, the balloon 430 is adapted to seat at the location and engage the circumferential region of tissue with the needles 440 penetrating therein. By injecting the material 414 through the needles in a sufficient volume and manner, their injectate will sufficiently inject along the circumference and thereby the circumferential conduction block may be formed.

It is to be appreciated that the conduction block formed by such a device and in similar manner may not be absolute or complete and still provide beneficial results. In one regard, transecting a portion of such a region of tissue may be sufficient to block an arrhythmic conduction path therethrough, such as across “fingers” of cardiac tissue that have been observed to extend up from atria and into the base of pulmonary veins. In addition, such balloon designs that have insufficient needle coverage to provide for overlap between their injectates may be partially rotated one or more times for better circumferential coverage and overlapping. Notwithstanding the foregoing, a complete or substantially complete circumferential conduction block at such pulmonary vein ostial location is considered a highly beneficial embodiment and optimal result in many cases. In fact, by providing such conduction block at such location of each pulmonary vein, atrial fibrillation may be cured without the need for mapping so extensively to identify which specific vessel houses a focal origin of such arrhythmia. While other such procedures using ablation techniques has been previously suggested, by removing the need for ablation according to the present invention, such empirical treatment modality involving all pulmonary veins may become in fact an appropriate choice for AFIB patient care.

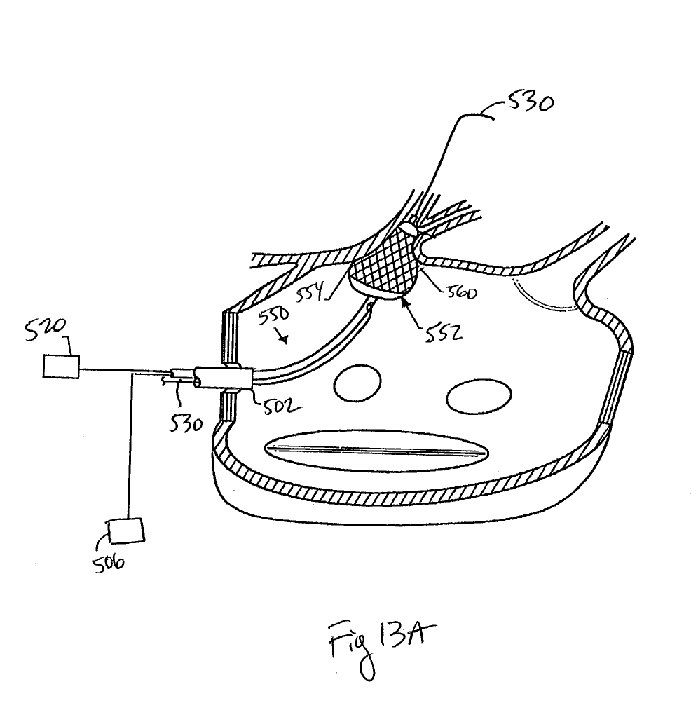

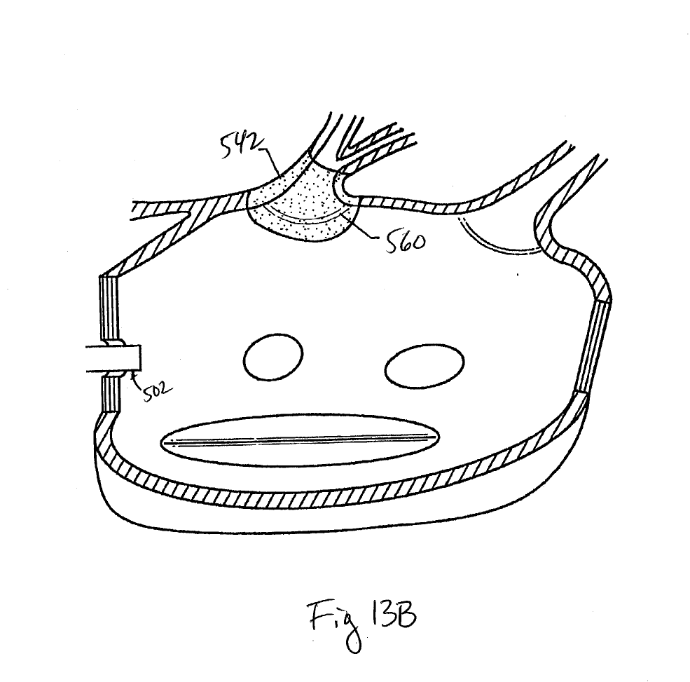

Various further enhancements or modifications of the device just described by reference to FIGS. 8A-9 may be made. For example, a deflectable tip design shown in FIG. 10 may be used wherein catheter 460 has a distal end portion 468 with a balloon 466 that is deflectable by manipulating actuator 464. Pull wire designs for example may be employed to achieve this embodiment. In another embodiment shown in FIG. 11, a catheter 470 has a guidewire tracking mechanism via an internal lumen that rides over a guidewire 480 so that distal end portion 478 and balloon 476 may be delivered to the pulmonary vein where the guidewire 480 is seated. Standard forms of guidewire coupling, e.g. using a hemostatic valve for example shown schematically at coupler 474 in FIG. 11, may be used.

In further exemplary modifications, needles may be replaced by other modes for delivering the desired material, such as through walls of porous membranes forming such a circumferential band. Other devices than a balloon may be used as well, such as expandable members such as cages, or other devices such as loop-shaped elongate members that may be configured with appropriate dimension to form the desired circumferential block. Moreover, other blocks than circumferential blocks may be made and still provide benefit without departing from the intended scope hereunder. In one regard, other conduction blocks may be done such as similar to the “maze” procedure and using similar techniques to those previously described using ablation technology.

The present invention is described herein by reference to several highly beneficial embodiments that provide conduction blocks in hearts, generally without without substantially ablating cardiac tissue. It is to be appreciated that the terms “without substantially ablating”, “substantially non-ablative,” or terms of similar import, are intended to mean that the primary mechanism of action is not ablation of tissue, and that the majority of tissue is not ablated at the location of material delivery. However, it is also to be considered that any material being delivered into a tissue may result in some attributable cell death. For example, the pressure of injection, or even the needle penetration itself, may be responsible for killing some cells, but such is not the mechanism by which conduction block is primarily achieved. In a similar regard, at some level it may be the case that all materials have some toxicity to all cells. However, a material is herein considered substantially non-ablative with respect to cardiac cells if such material does not cause substantial ablation as delivered, and cardiac cells can generally survive in the presence of such material in such delivered quantities.

It is also contemplated that cell delivery according to the invention may result in certain circumstances in substantial cell death in, or subsequent apoptosis of, the original cardiac cells in the region of tissue where delivery is performed, but such original cells are replaced by the transplanted cells. The result of such circumstance remains beneficial, as the structure remains cellular as a tissue and considered preferred over a scarred, damaged area as would result from classic ablation techniques. Moreover, it is considered generally beneficial even in the event of forming an ablative conduction block with the material delivery to do so with a material that is not in itself considered ablative to cardiac cells. In one regard, after forming the conduction block such material remaining in the region, and perhaps under active or passive transport in the area, is not continuing along an ablative or toxic course. Thus, in this sense for example, “non-ablative” may be considered in some regards to relate to cellular toxicity in the cardiac tissue.

In addition, despite the significant benefit provided according to the various aspects of the invention for non-ablative conduction blocks, further embodiments may also include ablative modes, such as for example by combining cell or fibrin glue delivery with ablation, either concurrently or serially.

Other specialized tools may be made for particular needs related to certain localized arrhythmias. As would be considered generally illustrated by the varied embodiments provided generally in the FIGS for example, a contact member is typically provided in the exemplary cardiac delivery system to contact the tissue at the target location and provide the required material delivery there. As generally illustrated by FIGS. 1-7B or 15 according to one of ordinary skill, certain needle or “end-hole” injection delivery catheters may be used in certain instances to inject the conduction block material at generally a single location, such as to insulate a focal source of arrhythmia, such as in a pulmonary vein subsequent to or contemporaneous with finding its location via mapping. In such circumstance, a catheter providing needle or end-hole infusion in combination with a tip mapping electrode may be used for example. Certain more complex “needle” injection devices have been disclosed, such as for example using screw needles with multiple ports along the screw shank, or the needle devices provided herein with multiple adjacent needles intended to provide localized mixing in tissues (e.g. FIG. 6). Nevertheless, these are generally considered “point” delivery devices to the extent the intended injection is into one localized site along the cardiac tissue structure wall.

In contrast, the embodiments of FIGS. 8A-11 provide general illustration according to one of ordinary skill that such delivery may be beneficially provided along a predetermined pattern of tissue along the respective cardiac tissue structure (e.g. wall) beyond a single injection site as would result from such needle or end-hole devices. More specifically, in order to create the necessary conduction blocks to treat many varied types of arrhythmias, it is often desired to provide the conduction block along a particular patterned region of tissue at a location associated with the arrhythmia. Thus the delivery catheter desired to achieve such block would be suitably adapted to deliver the non-ablative material along such patterned region. Such patterned delivery and resulting conduction block generally provides pre-determined geometry with varied dimensions (e.g. shapes having length, width, arcs, etc.), and may be for example elongated, such as linear or curvilinear, such as for example via shapeable, e.g. deflectable, or shaped elongated contact members. Other specific examples of desired patterns may be employed by combining multiple discretely patterned conduction blocks to achieve an overall patterned effect, such as for example similar to complex lesion patterns such as previously disclosed Cox-Maze type that provides a “box” encircling the pulmonary veins on the posterior left atrial wall (and often including an additional conduction block from the box to another cardiac structure providing conduction terminus, e.g. mitral valve or septum). Other examples include substantially circumferential conduction blocks as herein described for example for use at the base of pulmonary veins (e.g. FIGS. 8A-11).

Moreover, similar patterns may be used in different locations to provide conduction blocks against different arrhythmic pathways. For example, circumferential patterns used for pulmonary vein isolation may also be used to isolate atrial appendages, or at or adjacent to the valves to isolate atrial from ventricular conduction. While similar structures may be used to achieve similar patterns of conduction block in these locations, various modifications may be required to perform such activity in these different locations that may present unique access challenges or anatomical/dimensional characteristics.

It should also be appreciated that other modifications may be made to achieve similar objectives. For example, contact members such as cages, balloons, screw or needle anchors, may be used in order to anchor a delivery assembly in place so that needles or other injection or delivery members may be then extended from a position along the delivery catheter to another location adjacent to the contact member. In another regard, it is to be appreciated that contact members may include the needles themselves, and multiple needles may be employed in a spaced fashion over a pattern for delivery, allowing for the injection and subsequent diffusion or other transport mechanisms in the tissue to close the gaps and complete the pattern as one example of an equivalent approach to continuous, uninterrupted contact of a delivery member over that pattern. In other words, “contacting” a patterned region of tissue is considered contextual to the particular embodiment or application, and may be substantially continuous and uninterrupted contact in certain circumstances, or in others may have interruptions that are considered insignificant in the context of the anatomy or more general use.

For the purpose of further illustration, other more specific examples of delivery devices and methods that may be modified according to this disclosure to achieve the various objectives of the present invention are variously disclosed in one or more of the following issued U.S. patent references: U.S. Pat. No. 5,722,403 to McGee et al.; U.S. Pat. No. 5,797,903 to Swanson et al.; U.S. Pat. No. 5,885,278 to Fleishman; U.S. Pat. No. 5,938,660 to Swartz et al; U.S. Pat. No. 5,971,983 to Lesh; U.S. Pat. No. 6,012,457 to Lesh; U.S. Pat. No. 6,024,740 to Lesh et al.; U.S. Pat. No. 6,071,279 to Whayne et al.; U.S. Pat. No. 6,117,101 to Diederich et al.; U.S. Pat. No. 6,164,283 to Lesh; U.S. Pat. No. 6,214,002 to Fleischman et al.; U.S. Pat. No. 6,241,754 to Swanson et al.; U.S. Pat. No. 6,245,064 to Lesh et al.; U.S. Pat. No. 6,254,599 to Lesh et al.; U.S. Pat. No. 6,305,378 to Lesh; U.S. Pat. No. 6,371,955 to Fuimaono et al.; U.S. Pat. No. 6,383,151 to Diederich et al.; U.S. Pat. No. 6,416,511 to Lesh et al.; U.S. Pat. No. 6,471,697 to Lesh; U.S. Pat. No. 6,500,174 to Maguire et al.; U.S. Pat. No. 6,502,576 to Lesh; U.S. Pat. No. 6,514,249 to Maguire et al.; U.S. Pat. No. 6,522,930 to Schaer et al.; U.S. Pat. No. 6,527,769 to Langberg et al.; U.S. Pat. No. 6,547,788 to Maguire et al. The disclosures of these references are herein incorporated in their entirety by reference thereto.

To the extent these references variously relate to ablating tissue, the intended locations and patterns of conduction blocks, and therapeutic uses, and furthermore general delivery modalities, are considered useful according to further embodiments of the present invention to the extent modified for delivering non-ablative conduction block material or otherwise cellular transplantation into the cardiac tissue structures. For example, where ablation devices are disclosed, various related elements such as ablation electrodes, transducers, optical assemblies, or the like, would be replaced with suitable elements for injecting the materials of the type described herein. Other related elements such as ablation actuators, e.g. power sources, would be replaced with suitable sources of injectable material, and luminal structures of the delivery assemblies may be also suitably modified to provide for such injection to replace the prior modes of coupling such as electrical leads etc. Moreover, to the extent delivery of ablative fluids such as alcohol may be described by such previously disclosed systems and methods, such may be replaced by the materials and novel methods described herein according to still further embodiments of the invention.

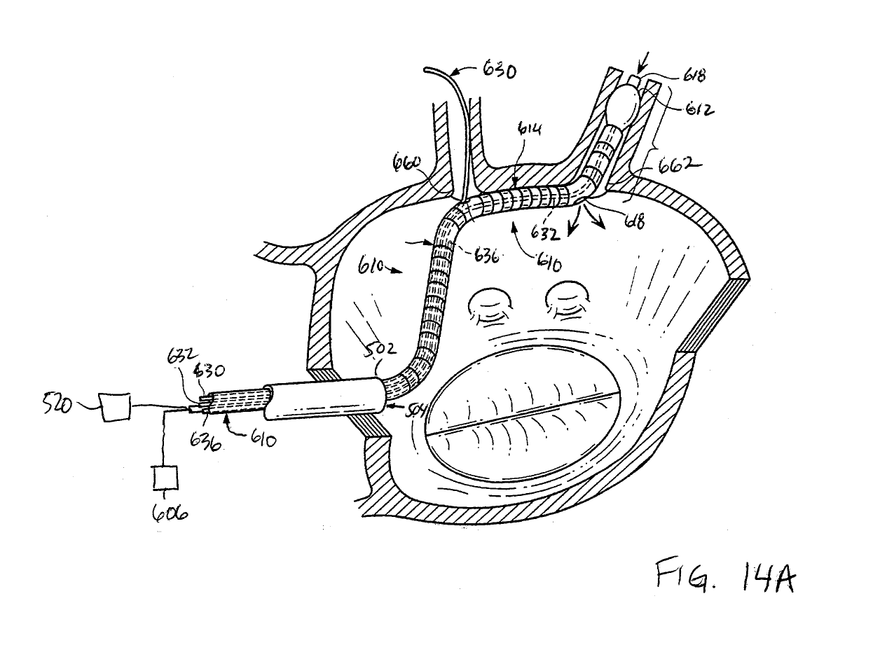

For further illustration, the following reference to FIGS. 12A-D and 13A-B provides such modification to certain embodiments of issued U.S. Pat. No. 6,012,457 to Lesh in order to provide for patterned conduction blocks according to the present invention for the purpose of pulmonary vein isolation as also previously illustrated by the embodiments above by reference to FIGS. 9-11.

More specifically, FIGS. 12A-D show a system 500 using a transeptal procedure via a transeptal sheath 502 providing a delivery lumen 504 into the left atrium of a heart in a patient. Delivery catheter 510 includes an expandable balloon 512 that is adjusted by an inflation device 504 (e.g. source of fluid) into a radially expanded configuration with an expanded outer diameter OD along a working length L that is engaged to a circumferential region of tissue at a location where a pulmonary vein extends from an atrium. A circumferential band 514 encircles the balloon 512 with a width w less than the working length L and is adapted to couple to source of material 520, shown schematically in FIG. 12A. Circumferential band 514 may carry a circumferential array of needles as previously described above, or may be porous, etc. to deliver the material that forms the conduction block.

The delivery catheter 510 shown is of a particular guidewire tracking type similar to shown and described by reference to FIG. 11, and in this particular illustrative variation is more specifically of the “rapid exchange” or “monorail” type. In other words, a lumen 518 is provided that tracks over a guidewire 530 over principally only a distal end portion of the catheter 510 that includes the balloon assembly 512. As shown in FIG. 12B, lumen 518 extends between distal port 517 and proximal port 519 on opposite sides of balloon 512. By withdrawing guidewire 530 after using it as a rail to deliver the balloon 512 to the pulmonary vein location for forming the conduction block, blood perfusion from the pulmonary vein into the atrium may be provided during balloon inflation, as shown in FIG. 12C.

Thereafter a further variation shown provides a proximal extension of lumen 518 along catheter 510 allows replacement of the guidewire 530 back through the catheter 510 for further “over the wire” use, such as for forming conduction block in a subsequent region of tissue where another pulmonary vein extends from the atrium. A resultant, illustrative conduction block 540 is formed with the material delivered along circumferential band 514, as shown in partially cross-sectioned view in FIG. 12D. This patterned block 540 may be illustrative of a complete circumferential pattern for the conduction block, or may be arcuate over only a portion of the circumference where shown. Further to that FIG. 12D, the guidewire 530 is further shown extended into a subsequent pulmonary vein for the next conduction block procedure where it extends from the atrium, if so desired.

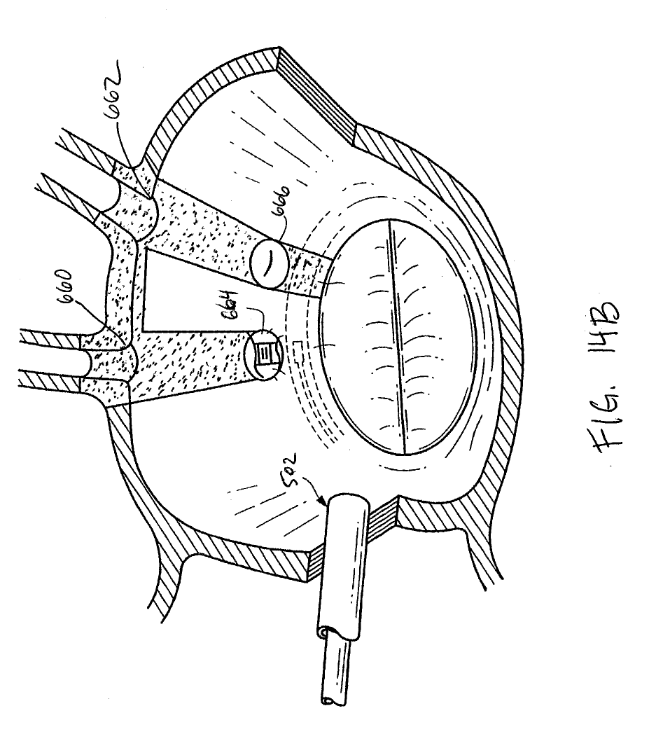

For still further illustration, FIG. 13A shows delivery catheter 550 as a modified form of catheter 510 shown in FIG. 12A, with a balloon 552 having in one regard a circumferential band 552 spanning a larger width for material delivery over the circumferential pattern. This provides a more extensive conduction block 542 (FIG. 13B) than according to the previous variation, covering tissue at ostium 560, as well as in a circumferential region of tissue above ostium 560 within the pulmonary vein, and circumferential region on the other side of ostium 560 immediately surrounding the ostium 560. Again, this may be completely circumferential, or arcuate over only a portion of the circumference, as desired for the particular arrhythmia treatment. Or, the device and/or method may be modified to provide the circumferential conduction block at only certain of these regions sufficient to isolate or cure a focus of arrhythmia.

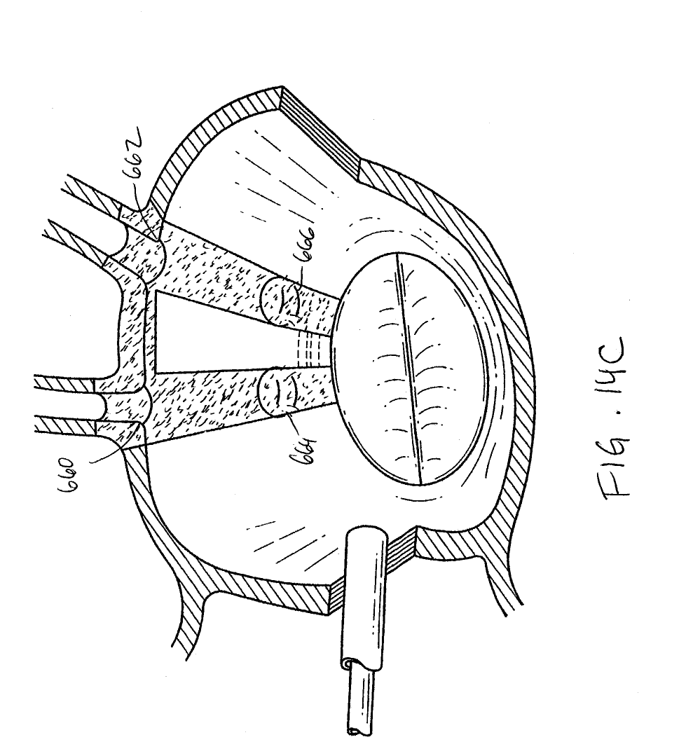

Still further examples are provided by reference to FIGS. 14A-C which respectively modify certain systems and methods disclosed in U.S. Pat. No. 5,971,983 to Lesh to deliver material for forming elongated, e.g. substantially linear or curvilinear, conduction blocks in a procedure similar to a modified “Cox-Maze” type method of forming an integrated network of conduction block segments to compartmentalize the posterior left atrial wall, and in particular the region bound by the pulmonary veins.

More specifically, a source of material 520 is coupled to a delivery catheter 610 that is delivered transeptally through a lumen 504 of a transeptal delivery sheath 502 and over two guidewires 630,632 in a manner adapted to drape catheter 510 between two adjacent pulmonary vein ostia 660,662, respectively engaged by those guidewires 630,632. A balloon 612 is coupled to inflation source 606, but contrary to other previous embodiments above functions primarily as an anchor to engage a pulmonary vein above ostium 662 and stabilizes delivery catheter 610 in position during delivery of material to form the conduction block. As shown before, guidewire 632 is shown withdrawn after delivery of the delivery catheter into the respective pulmonary vein in order to provide perfusion via guidewire lumen 618 while balloon 612 is inflated. However, as before, such perfusion capability may not be required, or may be suitable over the guidewire through the lumen without requiring proximal withdrawal.

According to this assembly, an elongated patterned region 614 extending between pulmonary vein ostia 660,662 is adapted to deliver material according to the invention from source 520 along that pattern to form a conduction block there. Bands are designated along the region 614 to schematically illustrate for example where a plurality of spaced needle injectors may be located to provide the patterned conduction block. Other regions are shown to also include such schematic bands, and may also be adapted to deliver material for conduction block formation.

A more advanced mode of forming the modified “Maze” type of conduction block pattern is shown in FIG. 14B after forming conduction blocks between pulmonary vein ostia 660,662, and between ostia 660,664, and between ostia 662,666. A further conduction block is shown between lower left ostium 666 and the mitral valve annulus to provide termination at a non-conductive structure to close the loop from otherwise pro-arrhythmia affects that could result in the atrium via a circular reentrant pathway around the pulmonary veins. FIG. 14B further illustrates in shadow delivery of material via the coronary sinus, a mode illustrative of transvascular delivery modes and devices according to further variations of the invention. A reference device may be placed in a pulmonary vein which may be used to assist in positioning the coronary sinus delivery catheter, as shown schematically within ostium 664 in FIG. 14B. In any event, a further modified overall pattern of conduction block is further shown in FIG. 14C, which may be formed in many varied specific modes than those specifically disclosed here for simplicity of illustration without departing from the intended scope of the invention.

Notwithstanding the substantial benefit that may be gained from such specialized tools and techniques to meet particular needs, it is to be considered that such particular adaptations for forming non-ablative conduction blocks, or otherwise conducting cell therapy for treating or preventing cardiac arrhythmias, are not to be considered limiting to the various broad aspects of the present invention.

### EXAMPLES

The following is a summary of certain specific examples of experiments that have been conducted and is being provided in order to provide a further understanding of various aspects of the present invention as described by reference to the Summary of the Invention and embodiments described above, and by further reference to the Figures in general.

### Example 1

Coupling requirements for successful impulse propagation with skeletal myocytes transplanted in myocardium have been determined by computer modeling as follows in order to determine whether transplanted myocytes can propagate electrical impulses within the myocardium.

The methods according to this example use computer modeling, which constructed theoretical strands of skeletal and mixed skeletal and ventricular myocytes.

The ventricular cells were an adaptation of the dynamic Luo Rudy ventricular cell formulation. Results according to this computer modeling study were as follows. In the mixed strand model, cardiac to skeletal coupling requirements were similar to cardiac-cardiac requirements. In contrast, skeletal to cardiac propagation failed at 300 nS, consistent with the need for a high degree of coupling. According to these results, conditions which decrease intercellular coupling appear to have a marked decrease on transmission between transplanted skeletal cells and the adjoining myocardium. Such effect has been observed to present risk of highly deleterious results when treating hearts in normal sinus rhythm, as the normal propagation of conduction may be dismantled.

However, the present invention contemplates localized use of such transplanted skeletal cells into areas of cardiac cells where conduction is irregular, such as re-entrant arrhythmia pathways. In this unique setting and environment of use, the decreased transmission of conduction arising from injecting cells of this or similar type into the cardiac tissues along such arrhythmia pathways becomes a potent mode for blocking and thus treating such related arrhythmias.

### Example 2

To assess the electrophysiologic consequences of skeletal muscle transplantation into the myocardium, an in vivo model was used to assess cardiac conduction. The feasibility of gene transfer to specific areas of the cardiac conduction system has been previously demonstrated (Lee et al. 1198 PACE 21-II: 606; Gallinghouse et al. November 1996 Am Heart Assoc.; U.S. Pat. No. 6,059,726). For example, the highly efficient and specifically localized expression of recombinant beta galactosidase in the AV node of rats and pigs has been described. The accuracy and reproducibility of AV nodal injections has been validated by the production of AV block in rats (Lee et al. 1998 J Appl Physiol. 85(2): 758-763). As an electrically insulated conduit for electrical transmission between the atrium and the ventricle, the AV conduction axis is in a strategic position for the study of cardiac electrophysiology.

To observe the effect of skeletal muscle transplantation on conduction and in particular regarding the electrophysiologic properties of AV nodes, a rat model for AV node injections was utilized (Lee et al. 1998 J Appl Physiol. 85(2): 758-763). Animals were chemically denervated (using atropine and propranolol to inhibit the influence of autonomic nervous system) and studied with right atrial overdrive pacing and atrial programmed extra-stimulation, both pre-injection and at the time of sacrifice. Surface ECG PR intervals were measured, together with AV nodal block cycle length (AVBCL) (the rate at which AV conduction becomes sequentially longer, then fails to conduct) and effective refractory period (ERP) (the coupling interval at which an atrial extrastimulus fails to conduct through the AV node). A single injection of skeletal myoblasts (1×10⁵, 15 ul) or vehicle was injected into the AVN of rats (n=8).

Electrophysiologic properties of the AV junction were significantly altered in animals with transplantation of skeletal myoblasts. Significant alterations in the Wenkebach cycle length (70.0±4.4 vs 57.0±5.0 msec; p<0.01) and AV nodal refractory period (113.8±5.6 vs 87.0±6.2 msec; p<0.005) were recorded in the skeletal myoblast injected rats as compared to control animals. Histological examination of the AVN revealed that approximately 10% of the AVN was involved with minimal to no inflammation. Histologically the AV conduction axis appeared normal in control vehicle injections. Interestingly, the PR interval did not significantly change, reflecting the insensitivity of surface EKG markers for cardiac conduction properties.

These results add further evidence that transplanted skeletal myoblasts (even when involving a small portion of the AVN) alters cardiac conduction and may lead to areas of slow conduction or conduction block. Therefore, as the skeletal myoblasts differentiate into myotubes and lose their ability to form gap junctions, the ability to propagate electrical impulses decrease.

Such loss of electrical impulse propagation, e.g. via gap junction loss as just demonstrated in this study, has been previously suggested to represent an adverse outcome to the desired result of treating damaged cardiac tissue via cell therapy by increasing conductivity and/or contractility. In particular with respect to AV node treatments previously posited, such decrease on electrical propagation to the extent of forming conduction blocks has not been previously suggested to be a desired result.

However, the present invention contemplates localized use of such transplanted skeletal cells into areas of cardiac cells where conduction is irregular, such as re-entrant arrhythmia pathways. In this unique setting and environment of use, the decreased transmission of conduction arising from injecting cells of this or similar type into the cardiac tissues along such arrhythmia pathways becomes a potent mode for blocking and thus treating such related arrhythmias.

### Example 3

In this study skeletal muscle was chosen as a test form of cell therapy for transplantation into the myocardium in arrhythmic animals to observe for antiarrhythmic effects.

The materials and methods used according to this study were as follows. Neonatal skeletal myoblasts were isolated as previously described by enzymatic dispersion from 2-5 days old Sprague Dawley neonatal rats and cultured as previously described (Rando, T., and Blau, H. M. (1994), J. Cell Biol. 125, 1275-1287). After isolation, cells were cultured with growth medium (GM) (80% F-10 medium (GIBCO BRL), 20% FBS (HyClone Laboratories, Inc.), penicillin G 100U/ml and streptomycin 100 ug/ml, bFGF 2.5 ng/ml (human, Promega Corp)). Skeletal myoblasts were maintained in GM medium in humidified 95% air and 5% CO₂ until used for transplantation.

Sprague-Dawley rats underwent 30 minutes of left coronary artery occlusion and 2 hours of reperfusion. One week following the creation of a myocardial infarction (MI) the rats were divided into two groups. Group 1 (n=7) received two injections (25 ul/injection) of vehicle control (PBS with 0.5% BSA) and Group 2 (n=5) received two injections (25 ul/injection) of rat skeletal myoblasts (total amount of cells: 5×10⁶). A third group of animals (Group 3) was added. Group 3 animals underwent the transplantation of skeletal myoblasts (1.5×10⁶) without an MI. Animals were survived. 5-6 weeks post-MI/cell injection, rats underwent programmed ventricular stimulation and ventricular fibrillation threshold testing. Following the completion of the pacing protocols, rat hearts were harvested for histology.

For this particular illustrative experiment, we use a 30 gauge needle to inject the cells in a single injection via a thoracotamy with direct vision of the heart. The location of injection was based upon results of a previous study, wherein another group of animals underwent 30 minutes of left coronary artery occlusion and 2 hours of reperfusion. After 5-6 weeks, the animals were sacrificed and the hearts isolated and perfused in a Langendorf preparation. Optical mapping was performed which demonstrated a re-entry circuit following the induction of ventricular tachycardia. The location of cell injections for the present study thus was chosen to include the border zone to interrupt such re-entry circuit.

Before sacrifice, ventricular programmed stimulation was performed by applying the pacing electrode on the right ventricle. The pacing protocol consisted of pacing the right ventricle with a train of 8 beats (cycle length of 140 ms) with up to three extra stimuli. Sustained ventricular tachycardia (VT) was defined as VT persisting more than 10 seconds and requiring cardioversion to sinus rhythm. Non-sustained VT (NSVT) was defined as lasting less than 10 seconds and self-limited.

Ventricular fibrillation thresholds (VFT) were obtained by placing the pacing electrode on the right ventricle. Burst pacing (50/sec for 2 sec) was applied and intensified by 0.1 mA each time using a Stimulator (Model DTU, Bloom Associates, LTD, Reading, Pa.). The average threshold of VF from three parts of the right ventricle was used as the electrical intensity which induced VF.

Observation of the test subjects yielded the following results shown in Tables 1 and 2:

TABLE 1
Myoblast Transplantation Effects on VT

| | NSVT | VT | No VT |
|---|---|---|---|
| Group 1 (MI + vehicle) | 1 | 6 | 0 |
| Group 2 (MI + myoblasts) | 2 | 0 | 3 |
| Group 3 (No MI + myoblasts) | 0 | 0 | 4 |

TABLE 2
Myoblast Transplantation Effects on VFT

| | VFT (mA) |
|---|---|
| Group 1 (MI + vehicle) | 1.2 ± 0.7 |
| Group 2 (myoblasts) | 3.3 ± 1.8 |

Conduction block was inferentially observed as the optical mapping studies demonstrated a re-entry pattern and the cell delivery prevented sustained VT.

According to the foregoing observations and results of this study, transplantation of skeletal myoblasts into ventricle wall tissue completely prevent sustainable VT in all subjects receiving the cell therapy. In another regard, transplantation of skeletal myoblasts increases the amount of energy required to induce VF versus untreated myocardium. Accordingly, transplantation of myoblasts into cardiac tissue of the ventricle wall provides a potent anti-arrhythmic effect on such tissue. Moreover, the myoblast injections into regions associated with reentry circuits demonstrated anti-arrhythmic effects attributable to conduction block.

The observations, results, and conclusions related to the foregoing study are considered exemplary of cell therapy in general as a potent agent for preventing and treating arrhythmia, and more specifically creating conduction blocks without ablating tissue. Skeletal myoblasts were used as the chosen test sample, and are considered a highly beneficial mode according to the present invention as shown in this study. However, as mentioned above, such use of myoblasts are considered illustrative of a class of cells whose introduction into the cardiac tissue structures intervenes sufficiently to arrhythmic conduction pathways to either create a block or slow the transduction sufficient to reduce the overall effect on sinus rhythm. Such class includes for example other suitable substitute types of cells for providing similar therapy or prophylaxis of cardiac arrhythmias, such as for example stem cells or fibroblasts. Accordingly, in particular with regard to previous cell therapy disclosures intended to primarily increase cardiac conduction such as by modifying activity of cells being delivered, the invention should be considered to broadly encompass cell therapy adapted to block conduction of arrhythmias in tissues associated with cardiac chambers.

Moreover, ventricular arrhythmias were used as the chosen test environment to observe for such anti-arrhythmic effects. Accordingly, a highly beneficial method for treating ventricular arrhythmias, and in particular ventricular fibrillation and tachycardia, has been shown and is considered a beneficial aspect of the invention. However, it is further contemplated that such use is also illustrative of modes for treating arrhythmias in general, and other suitable substitute treatment modalities using cell therapy are contemplated. For example, arrhythmias of either or both ventricles may be treated or prevented using such cell therapy techniques. Still further, atrial arrhythmias such as atrial fibrillation may be treated or prevented. In general, the ability to use cell transplantation to block arrhythmic conduction pathways as illustrated in this present Example is considered applicable to such pathways of any or all the chambers.

Notwithstanding the foregoing, each cell type is considered unique and is therefore believed to provide particular aspects to be accounted for during use.

### Example 4

In this study, fibroblasts were used according to various aspects of the invention to observe the effects of their transplantation into cardiac tissue on cardiac arrhythmias.

The purpose of the study is to confirm that fibroblast transplantation into the myocardium effects myocardial remodeling and acts as an anti-arrhythmic agent in preventing ventricular tachycardia.

Dermal fibroblasts were prepared from the skin of fetal Fisher rats. Tissue fragments were digested for 30 minutes in 0.2 U/mL collagenase solution before being plated on collagen-coated dishes in DMEM with 10% FBS and Pen-Strep. The cells were grown at 37° C. in 5% CO₂ and passaged upon reaching ˜70% confluence, up to the fourth passage. Fibroblasts were selected using a differential adhesion method, where the mixed cell population was incubated for 15 minutes in culture conditions, during which time fibroblasts adhered to the culture plate and myoblasts remained in suspension to be replaced by fresh culture medium.

To verify purity of the fibroblast culture, immunohistochemistry was performed using antibodies to vimentin (1:20 dilution), an intermediate filament present in both myoblasts and fibroblasts, and desmin (1:100 dilution), a muscle-specific protein. Cell suspensions from fibroblast cultures were pipetted into chamber slides and cells were allowed to attach and spread overnight. They were fixed with 2% paraformaldehyde for 5 minutes, then 100% methanol at 0 degrees C. for another 5 minutes. After several PBS rinses and staining buffer blocking, the primary antibodies were added to separate chambers for one hour. (A pure myoblast culture was also used for a positive control for anti-desmin.) Secondary antibodies used were Cy3-conjugated anti-rabbit IgG (1:500 dilution) for the anti-desmin stains, and Cy3-conjugated anti-mouse IgG (1:200 dilution) for the anti-vimentin stains.

Fisher rats were subjected to 30 minutes of left coronary artery occlusion and 2 hours of reperfusion. One week following the creation of a myocardial infarction (MI) the rats were divided into two groups. Group 1 (n=8) received two injections (25 ul/injection) of vehicle control (PBS with 0.5% BSA) and Group 2 (n=8) received two injections (25 μl/injection) of rat fibroblasts (total amount of cells: 5×10⁶). A dose response was performed with at least 2 other doses of fibroblasts. Fibroblasts were isolated from a skin biopsy, amplified and reinjected into the rat from which the biopsy was taken thus avoiding rejection. Fibroblasts were stained with marker dyes such as BRDU, CFDA-SE or transfected with B-galactosidase to identify transplanted fibroblasts from cardiac fibroblasts. A third group of animals (Group 3, n=8) received transplantation of fibroblasts (1.5×10⁶) without an MI. Animals were survived and underwent echocardiography at week 1 and week 5. 5-6 weeks post-MI/cell injection, rats received programmed ventricular stimulation and ventricular fibrillation threshold testing. Following the completion of the pacing protocols, rat hearts were harvested for histology. MI size and distribution of transplanted fibroblast were determined by histological examination.

Ventricular programmed stimulation was performed by applying the pacing electrode on the right ventricle. The pacing protocol consisted of pacing the right ventricle with a train of 8 beats (cycle length of 140 ms) with up to three extrastimuli. Sustained ventricular tachycardia (VT) was defined as VT persisting more than 10 seconds and requiring cardioversion to sinus rhythm. Non-sustained VT (NSVT) was defined as lasting less than 10 seconds and self-limited.

Ventricular fibrillation thresholds (VFT) were obtained by placing the pacing electrode on the right ventricle. Burst pacing (50/sec for 2 sec) was applied and intensified by 0.1 mA each time using a Stimulator (Model DTU, Bloom Associates, LTD, Reading, Pa.). The average threshold of VF from three parts of the right ventricle were used as the electrical intensity which induced VF.

According to initial results per this protocol above, five (5) rats had no inducible VT, with average ventricular fibrillation threshold equal to 5.5 mA. However, in contrast to previous experiments of the Examples 2-3 above, this study only had 3 control animals which did not have inducible VT. In one regard, in contrast to the other studies above, this study used a different strain of rats.

Despite the absence of a useable control in this study showing unique results between the groups, it is believed that conduction blocks were formed by the fibroblasts in the treatment group rats based upon: (i) the myoblast experience of the prior examples above, (ii) per a further understanding of fibroblast activity as noted above, and (iii) in consideration of the results in this study showing no sustainable VT in treatment group rats. Confirmation of such belief merely requires reproducing such study in a manner yielding a better control (e.g. in a different animal strain).

### Example 5

The purpose of this study was to further confirm effects of fibroblast therapy on ventricular arrhythmogenicity in a rat model of ischemia-reperfusion, and more specifically confirm that fibroblast transplantation into the myocardium acts as an antiarrhythmic agent in preventing ventricular tachycardia.

Tissue engineering techniques utilizing skeletal myoblast transplantation for myocardial repair has gained increased attention with the demonstration that skeletal myoblasts survive and form contractile myofibers in normal and injured myocardium. However, the emphasis of myocardial repair has focused on the preservation of myocardial contractility with little attention given to the effects of tissue engineering on cardiac conduction or arrhythmogenesis.

Fibroblasts' electrophysiological properties are fairly consistent from one fibroblast to the next. Therefore, when fibroblasts are used to block VT, a higher degree of certainty exists that the same response will result from one batch/injection to the next. According to the foregoing, fibroblast transplantation into cardiac tissue structures should block conduction in a repeatable and predictable fashion. In contrast, skeletal muscle transplantation generally involves initial injection as myoblasts that differentiate into myotubes and myofibers which have significantly different conduction properties. Additionally, depending on how old the myoblasts are, they can vary in conduction properties. Therefore, following the injection of myoblasts, a heterogeneous milieu of cells may result in certain instances which may not provide the insulation properties desired for an effective conduction block. Nevertheless, myoblast therapy has been shown to generally provide effective anti-arrhythmic properties and an effective conduction block technique that is believed to be effective in many if not most cases. It is thus believed that, notwithstanding beneficial results observed with myoblast transplantation for forming conduction blocks, fibroblasts are believed to be particularly beneficial in certain regards.

According to this study, Fisher rats underwent 30 minutes of left coronary artery occlusion and 2 hours of reperfusion. One week following the creation of a myocardial infarction (MI) the rats were divided into two groups. Group 1 (n=14) received two injections (25 ul/injection) of vehicle control (PBS with 0.5% BSA) and Group 2 (n=11) received two injections (25 ul/injection) of rat fibroblasts (total amount of cells: 5×10⁶). 5-6 weeks cell injection, rats underwent programmed ventricular stimulation and ventricular fibrillation threshold testing.

Ventricular programmed stimulation was performed by applying the pacing electrode on the right ventricle. The pacing protocol consisted of pacing the right ventricle with a train of 8 beats (cycle length of 140 ms) with up to three extrastimuli. Sustained ventricular tachycardia (VT) was defined as VT persisting more than 10 seconds and requiring cardioversion to sinus rhythm. Non-sustained VT (NSVT) was defined as lasting less than 10 seconds and self-limited.

Ventricular fibrillation thresholds (VFT) was obtained by placing the pacing electrode on the right ventricle. Burst pacing (50/sec for 2 sec) was applied and intensified by 0.1 mA each time using a Stimulator (Model DTU, Bloom Associates, LTD, Reading, Pa.). The average threshold of VF from three parts of the right ventricle will be used as the electrical intensity which induced VF.

TABLE 3
Fibroblast Transplantation Effects on VT

| | total | sustained | unsustained |
|---|---|---|---|
| | number | VT | VT or no VT |
| Fibroblast group | 11 | 4 | 7 |
| BSA group | 14 | 13 | 1 |

P value (Chi Square test) < 0.003

TABLE 4
Fibroblast Transplantation Effects on VFT

| | total number | VF threshold (mA) |
|---|---|---|
| Fibroblast group | 11 | 3.76 ± 1.5 |
| BSA group | 14 | 1.70 ± 1.4 |

P value (T-test) < 0.002

According to the results observed and summarized in Tables 3 and 4 immediately above, fibroblast transplantation into a ventricle wall prevents ventricular tachycardia and increases the ventricular fibrillation threshold (in other words, it takes more energy to induce ventricular fibrillation).

It is to be further noted that two animals using the above protocol (ligation of the LAD to produce myocardial infarction) also underwent the injection of fibrin with fibroblasts. 5 weeks after the injection, programmed electrical stimulation was performed. No VT was induced. This preliminary result suggests that fibrin with fibroblasts can prevent ventricular arrhythmias.

### Example 6

In this study, the effects of injecting fibrin glue, an injectable biopolymer, into cardiac tissue structures were examined, with particular respect to providing an internal support and scaffold and whether it could improve cardiac function and increase infarct wall thickness following MI. Based upon such observations, further use in forming conduction blocks was explored.

A previously described rat ischemia reperfusion model was used in this study. Female Sprague-Dawley Rats (225-250 g) were anesthetized with ketamine (90 mg/kg) and xylazine (10 mg/kg). Under sterile technique, the rats were placed in supine position and the chest was cleaned and shaved. The chest was opened by performing a median sternotomy. Keeping the landmarks of the base of the left atrium and the interventricular groove in view, a single stitch of 7-0 Ticron suture was placed through the myocardium at a depth slightly greater than the perceived level of the left anterior descending portion (LAD) of the left coronary artery while taking care not to enter the ventricular chamber. The suture was tightened to occlude the LAD for 17 minutes and then removed to allow for reperfusion. The chest was then closed and the animal was allowed to recover for 1 week.

Myoblasts from the hind limb muscle of Sprague-Dawley neonatal rats (2-5 days old) were isolated and purified according to the following described procedure. Briefly, the hind limb was harvested under Phosphate buffered saline (PBS)-Penicillin/Streptomycin (PCN/Strep) and mechanically minced. The tissue was enzymatically dissociated with dispase and collagenase (Worthington) in Dulbecco's PBS (Sigma) for 45 minutes at 37° C. The resulting suspension was then passed through an 80 μm filter and the cells were collected by centrifugation. The cells were preplated for 10 minutes in order to isolate myoblasts from fibroblasts. The myoblast suspension was transferred to a collagen coated 100 mm polystyrene tissue culture dish (Corning Inc) and allowed to proliferate in growth media (80% Ham's F10C media, 20% fetal bovine serum, 1% PCN/Strep, 2.5 ng/ml recombinant human basic fibroblast growth factor) at 37° C. in a humidified atmosphere of 95% air plus 5% CO₂. Cultures were allowed to reach a confluency of 70-75% and passaged every 3-4 days (1:4 split).

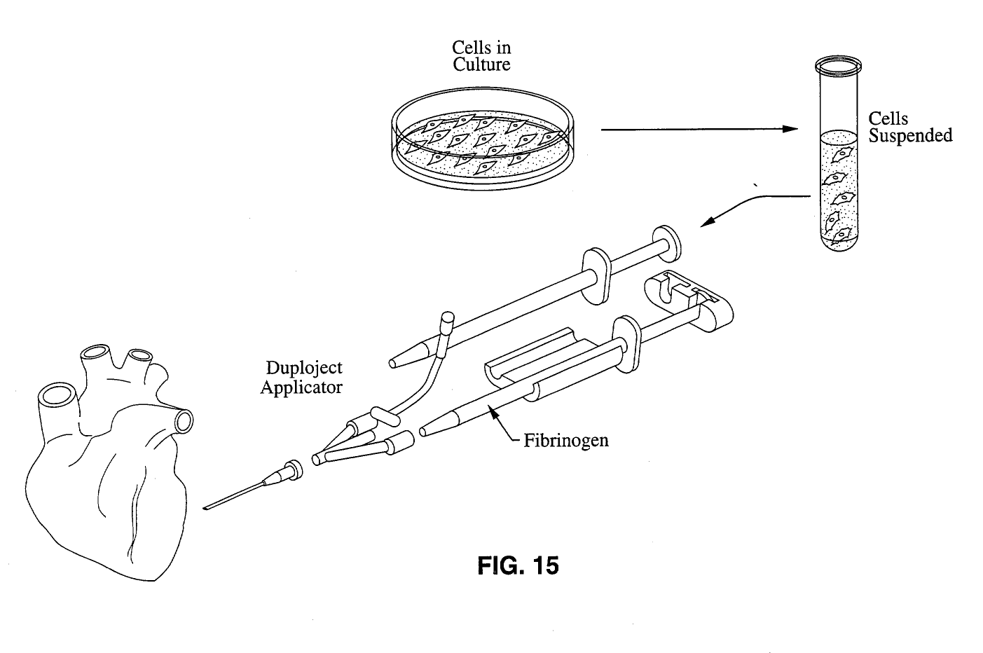

The fibrin glue used in this study was the commercially available Tisseel VH fibrin sealant (commercially available from Baxter). It is a two component system which remains liquid for several seconds before solidifying into a solid gel matrix. The first component consists of concentrated fibrinogen and aprotinin, a fibrinolysis inhibitor. The second is a mixture of Thrombin and CaCl₂. It is delivered through the supplied Duploject applicator, which holds the two components in separate syringes, respectively, and provides simultaneous mixing and delivery (as shown stepwise schematically in FIG. 15). The ratio of fibrinogen to thrombin components was 1:1.

Approximately 1 week after MI, either 0.5% bovine serum albumin (BSA) in 50 microliter PBS (control group), 50 microliter fibrin glue, 5×10⁶ myoblasts in 50 microliter 0.5% BSA, or 5×10⁶ myoblasts in 50 microliters fibrin glue was injected into the ischemic LV. Under sterile technique, the rats were anesthetized and the abdomen was opened from the xiphoid process to a left subaxillar level along the lower rib. The LV apex was exposed via a subdiaphragmatic incision, leaving the chest wall and sternum intact. Rats were randomized to either control or treatment groups and injections were made through a 30-guage needle into the ischemic LV. In the cells group, 5×10⁶ myoblasts were suspended in 50 microliter 0.5% BSA and injected into the myocardium. In the cells in fibrin group, 5×10⁶ myoblasts were suspended in 25 microliter of the thrombin component of the fibrin glue. The thrombin-cell mixture was simultaneously injected into the myocardium with 25 microliter of the fibrinogen component (FIG. 15). 25 microliter thrombin and 25 microliter fibrinogen was simultaneously injected into ischemic myocardium in the fibrin group. The diaphragm was sutured closed after suction of the chest cavity and the abdomen was subsequently closed

Transthoracic echocardiography was performed on all animals in conscious state approximately one week after MI (baseline echocardiogram), followed by control or treatment injections 1-2 days later. Then a follow-up echocardiogram was performed approximately 4 weeks later. The methodology of echocardiography used in this laboratory has been previously described. Other reports have demonstrated the accuracy and reproducibility of transthoracic echocardiography in rats with myocardial infarcts.

Briefly, the animals were shaved and placed in plastic DecapiCone restrainers (Braintree Scientific Inc.) in conscious state. A layer of acoustic coupling gel was applied to the thorax. Then the animal was placed in a prone or slightly lateral decubitus position. Echocardiography was performed using a 15-MHz linear array transducer system (Acuson Sequoia c256, Mountain View, Calif.). Care was taken to avoid excessive pressure on thorax, which could induce bradycardia. Two-dimensional images were obtained in both parasternal long and short axis views (at the papillary muscle level). Enhanced resolution imaging function (RES) was activated with a region of interest adjusted to heart size whenever possible. The gain was set for best imaging, and the compression was set at 70 dB. The images were acquired digitally and stored on magneto-optical disk (SONY EDM-230C).

Two criteria were used for imaging according to this particular experiment model. First, the short-axis view was given the criteria to demonstrate at least 80% of the endocardial and epicardial border. Second, the long-axis view was given the criteria to demonstrate the plane of mitral valve, where the annulus and the apex could be visualized. After adequate two-dimensional images were obtained, the M-mode cursor was positioned perpendicular to the ventricular anteroseptal wall (at the site of infarct) and the posterior wall, at the level of the papillary muscles. Wall thickness and left ventricular internal dimensions were measured according to the leading edge method of the American Society of Echocardiography. Fractional shortening (FS) as a measure of systolic function was calculated as FS (%)=[(LVIDd−LVIDs)/LVIDd]×100%, where LVID was the left ventricular internal dimension, d was diastole and s was systole. An echocardiographer blinded to the treatment group acquired the images and performed the data analysis. The accuracy and reproducibility of the technique have been reported in a previous study from this laboratory.

Approximately 4 weeks following the injection surgeries, the rats were euthanized with a pentobarbital overdose (200 mg/kg). The hearts were rapidly excised and fresh frozen in Tissue Tek O.C.T. freezing medium. They were then sectioned into 5 micron slices and stained with hematoxylin and eosin (H&E). A subset of hearts from the cells group and cells in fibrin glue group were stained with the MY-32 clone (Sigma), which is directed against the skeletal fast isoform of myosin heavy chain (MHC), in order to label transplanted cells. A Cy-3 conjugated anti-mouse secondary antibody (Sigma) was used to visualize labeled cells. One 250 microliter sample of fibrin glue was also fresh frozen, sectioned into 5 micron slices and stained with H&E.

Data is presented as mean±standard deviation. The rat myocardial infarction model has been generally observed to have a high degree of variability, thus internal controls are implemented in order to evaluate treatment effects. Differences of fractional shortening and infarct wall thickness between measurements before and after injection were compared using a 2 tailed paired t test. Such differences were compared across treatment group using a one-way ANOVA with Bonferroni adjustment. Measurements after injection were also compared between groups using a one-way ANOVA with Bonferroni adjustment. Significance was accepted at P<0.05.

A total of 41 rats were used in this study. Six rats died during or immediately following the infarct surgery while one rat died during the injection surgery (cells in fibrin glue group). Post-injection surgery, there was 100% survival in all groups. Final echocardiography measurements were performed on 34 rats. The control group (n=7) was injected with 0.5% BSA, the fibrin group (n=6) was injected with fibrin glue, the cells group (n=6) was injected with 5×10⁶ myoblasts, and the cells in fibrin group (n=5) was injected with 5×10⁶ myoblasts in fibrin glue.

Echocardiography measurements were collected approximately one week post-MI (prior to injection surgery) and approximately four weeks following the injection surgery in order to determine the effects of fibrin glue, myoblasts, and a combination of the two on LV function and infarct wall thickness. Results are provided in the following Table 5:

TABLE 5
Echocardiography Data

| | Before | 4 Weeks Post- | |
|---|---|---|---|
| | Injection | Injection | P |
| Fractional shortening, % | | | |
| Control group | 45 ± 8 | 22 ± 6 | 0.0005 |
| Fibrin group | 26 ± 5 | 23 ± 8 | 0.18 |
| Cells group | 29 ± 14 | 28 ± 2 | 0.89 |
| Cells in fibrin group | 42 ± 10 | 33 ± 6 | 0.19 |
| Infarct wall thickness, cm | | | |
| Control group | 0.29 ± 0.08 | 0.24 ± 0.04 | 0.02 |
| Fibrin group | 0.26 ± 0.04 | 0.23 ± 0.06 | 0.40 |
| Cells group | 0.30 ± 0.08 | 0.26 ± 0.06 | 0.44 |
| Cells in fibrin group | 0.30 ± 0.04 | 0.32 ± 0.02 | 0.43 |

As typical of post-MI progression, the control group exhibited a deterioration of LV function and thinning of the infarct wall. After four weeks there was significant deterioration in FS (P=0.0005) as well as a significant decrease in infarct wall thickness (P=0.02) (Table 5, control group).

In contrast, injection of fibrin glue alone, myoblasts alone, and myoblasts in fibrin glue resulted in the preservation of FS and infarct wall thickness. FS for the fibrin group, cells group, and cells in fibrin group did not significantly decrease by P-values of 0.18, 0.89, and 0.19 respectively (Table 5). In addition, there was no significant difference in infarct wall thickness for all treatment groups (P=0.40, 0.44, 0.43 respectively) (Table 5). Differences between before injection and post-injection FS and infarct wall thickness were compared among treatment groups. No significant difference was observed (P=0.52 and P=0.56 respectively), thus indicating that no single treatment was more effective than the others. A comparison of infarct wall thickness among all groups four weeks after injection demonstrates that the wall thickness of the cells in fibrin group is statistically greater than the control (P=0.009) and fibrin groups (P=0.04); however, due to the high degree of variability among infarcts as previously stated, it is more meaningful to use data comparing internal controls.

Fibrin glue is generally observed to form a fibril and porous structure containing fibrils and pores having diameter greater than 2 microns, and is generally termed a coarse gel. Examination of H&E stained heart sections revealed extensive transmural MIs in all groups. In the infarct region, native cardiomyocytes were replaced by fibrillar collagenous scar tissue. At four weeks after injection, the fibrin glue was completely degraded and not visible. Immunostaining for skeletal fast MHC demonstrated that transplanted cells in both the cells group and cells in fibrin group were viable four weeks post-injection and distributed throughout the infarct scar. The transplanted myoblasts in the infarct wall of a heart that was injected with myoblasts in fibrin glue were observed to be aligned in a parallel orientation.

Additionally, cell survival within the infarcted myocardium was enhanced. The mean area covered by transplanted myoblasts was significantly greater when injected in the fibrin scaffold compared to injection in BSA (P=0.02). The myoblast area for cells injected in fibrin glue was 2.8±0.9 mm² while the area for cells injected in BSA was 1.4±0.5 mm². Transplanted myoblasts injected in BSA were most often found at the border of the infarct scar and not within the ischemic tissue. In contrast, myoblasts injected in fibrin glue were found both at the border and within the infarct scar. Cells transplanted in fibrin glue were often surrounding arterioles within the infarct scar.

Fibrin glue, though highly beneficial according to the embodiments of the study herein disclosed, is a biopolymer and thus is illustrative of other materials of similar composition or function in the environment of use that may be suitable substitutes, e.g. other biopolymers.

Fibrin glue is formed by the addition of thrombin to fibrinogen. Thrombin enzymatically cleaves fibrinogen which alters the charge and conformation of the molecule, forming a fibrin monomer. The fibrin monomers then proceed to aggregate forming the biopolymer fibrin. Fibrin is highly involved in wound healing in the body and in conjunction with platelets, is the basis of a clot. No adverse reactions were observed upon injection into the myocardium, including no delivery of clot to or from the heart. Fibrin is resorbed by enzymatic and phagocytic pathways, thus it was expected that no traces of fibrin would remain four weeks post-injection.

The results of the present study indicate that fibrin glue is useful as a support and/or tissue engineering scaffold to prevent LV remodeling and improve cardiac function following MI. Injection of fibrin glue alone as well as injection of skeletal myoblasts in fibrin glue attenuated any decrease in infarct wall thickness and fractional shortening following MI in rats. In accordance with other studies, we also found that injection of skeletal myoblasts alone was able to prevent negative remodeling of the infarcted LV and deterioration of LV function. Although the exact mechanism by which myoblasts preserve LV function is unknown, it is unlikely that it is from active force generation during systole since implanted myoblasts do not form gap junction with surrounding cardiomyocytes. It is believed that the attenuation of negative left ventricular remodeling by the myoblasts is the mechanism that preserves cardiac function. The myoblasts may serve as a wall support by increasing stiffness, or may simply affect remodeling by increasing wall thickness. The data according to this study further supports this. Injection of fibrin glue alone did not produce statistically different results from the injection of skeletal myoblasts, thus suggesting that the mechanism of action of the myoblasts is by preserving wall thickness and preventing deleterious ventricular remodeling, not from active force generation.

A recent study disclosed use of a polymer mesh for the intended purpose of acting as an external support to prevent LV dilation. Fibrin glue may act as an internal support to preserve cardiac function. During the initial stage in MI, matrix metalloproteases are upregulated which results in degradation of the extracellular matrix (ECM). This ECM degradation leads to weakening of the infarct wall and slippage of the myocytes leading to LV aneurysm. In addition, it has been disclosed that negative ventricular remodeling continues until the tensile strength of the collagen scar strengthens the infarct wall. By administering fibrin glue during the initial stage of an infarct, it may prevent remodeling by increasing the mechanical strength of the infarct before the collagen scar has had to time to fully develop. Furthermore, fibrin glue adheres to various substrates including collagen and cell surface receptors (predominately integrins) through covalent bonds, hydrogen and other electrostatic bonds, and mechanical interlocking. Therefore, it may prevent myocyte slippage and subsequent aneurysm by binding to the neighboring normal myocardium. Finally, injection of fibrin glue is also believed to result in an upregulation or release of certain growth factors such as angiogenic growth factors which may improve cardiac function.

In addition to providing an internal support, according to the data of this study it is believed that fibrin is useful as a tissue engineering scaffold in the myocardium. Injection of myoblasts in fibrin glue prevented infarct wall thinning and preserved cardiac function. The wall thickness of this group was also significantly greater than that of other groups. Several previous publications have disclosed delivering a variety of cell types including keratinocytes, fibroblasts, chondrocytes, urothelial cells, and corneal epithelial cells in a fibrin glue scaffold. The results according to the present study also indicate that fibrin glue is capable of delivering viable cells to the myocardium. Although it unlikely that unmodified skeletal myoblasts improve contractility, other cell types including fetal cardiomyocytes and adult bone marrow stem cells, which produce gap junctions in recipient hearts, could be delivered to the myocardium in fibrin glue with the aims of improving both contractility and preventing remodeling.

Another previous disclosure used a tissue engineering approach by delivering fetal cardiomyocytes in alginate scaffolds to the surface of the myocardium and reported preservation of cardiac function. Their results were most likely due to the transplantation of fetal cardiomyocytes and not to the external support of the scaffold due to its small size compared to the LV. The benefit of using fibrin glue as a scaffold is that it is injectable, thus requiring only a minimally invasive procedure in humans. In addition, the cells are delivered directly into the infarcted tissue instead of simply on the epicardial surface.

Notwithstanding the foregoing, and despite what specific mechanisms are in particular involved, the compound preparation, systems, and methods herein disclosed are nevertheless clearly shown to provide the intended results in treating certain cardiac conditions consistent with the various objects and aspects of the invention.

The results according to this study confirm that preparations and uses of fibrin glue according to the present invention provides a beneficial treatment for patients who suffer from MI. The study shows use of an injectable internal support and/or tissue engineering scaffold to prevent deleterious ventricular remodeling and deterioration of cardiac function. As a support, fibrin glue may be modified to tailor its mechanical properties for this particular application, which modifications are contemplated within the scope of the invention. An increase in thrombin or fibrinogen concentration results in an increase in tensile strength and Young's modulus. An increase in fibrinogen concentration will also decrease the degradation rate of the biopolymer. As a tissue engineering scaffold, fibrin glue is also capable of delivering proteins and plasmids and further embodiments contemplated hereunder use such mechanism to deliver both growth factors, either in protein or plasmid form, and cells to the myocardium.

According to the observations and results of the foregoing study, the present invention further contemplates use of fibrin glue agent, either alone or in combination with certain types of cells, as an injectable material for forming conduction block in cardiac tissue.

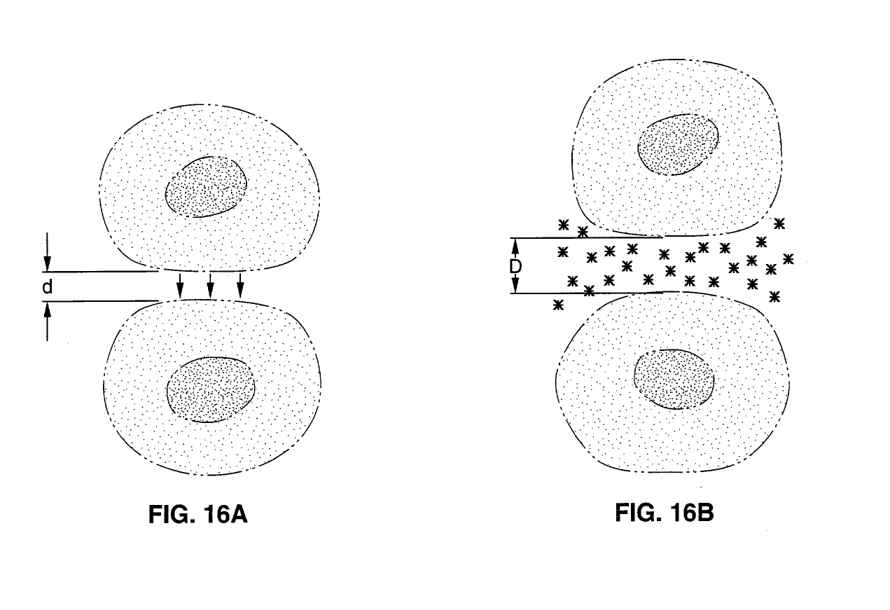

In addition to the mechanisms of action elsewhere herein described, it is further contemplated that injectable materials such as fibrin glue according to the invention may provide conduction block results at least in part by physically separating cells in the region of injection. For further illustration, FIGS. 16A-B show transition between a cellular matrix in an initial gap junction condition (FIG. 16A), and in a post-treatment condition wherein the spacing between cells is physically separated between an initial distance d to a larger, separated distance D (FIG. 16B). These separations may be sufficient to raise the action potential to stimulate conduction between cells to such level that conduction is blocked or otherwise retarded sufficiently to halt arrhythmia.

Notwithstanding certain theories and beliefs provided herein with respect to the mechanisms by which certain embodiments perform, it is to be appreciated that the use of certain materials and procedures to the extent they produce certain intended results are contemplated under the invention despite the actual mechanism by which the results are accomplished.

Various descriptions of materials provided herein may be in particular beneficial, such as for example various references to fibrin glue or related agent, or analogs or derivatives thereof. However, other suitable materials may be used in certain applications, either in combination or as substitutes for such particular materials mentioned. In one particular regard, where fibrin glue or related agents are herein described, it is further contemplated that collagen, or precursors or analogs or derivatives thereof, may also be used in such circumstances, in particular relation to forming conduction blocks or otherwise treating cardiac arrhythmias. Moreover, where collagen is thus included, precursor or analogs or derivatives thereof are further contemplated, such as for example structures that are metabolized or otherwise altered within the body to form collagen, or combination materials that react to form collagen, or material whose molecular structure varies insubstantially to that of collagen such that its activity is substantially similar thereto with respect to the intended uses contemplated herein (e.g. removing or altering non-functional groups with respect to such function). Such group of collagen and such precursors or analogs or derivatives thereof is herein referred to as a “collagen agent.” Similarly, reference herein to other forms of “agents”, such as for example “polymer agent” or “fibrin glue agent” may further include the actual final product, e.g. polymer or fibrin glue, respectively, or one or more respective precursor materials delivered together or in a coordinated manner to form the resulting material.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A system for treating a cardiac arrhythmia in a heart of a patient, comprising:
a cardiac delivery system; and
a source of material coupled to the cardiac delivery system;
wherein the delivery system is adapted to deliver a volume of the material from the source to a to region of tissue at a location associated with the cardiac arrhythmia and that includes cardiac cells;
wherein the material is characterized as being substantially non-ablative with respect to cardiac cells; and
wherein the material is adapted to form a conduction block in the region.

2. The system of claim 1, wherein the material comprises living cells.

3. The system of claim 2, wherein the living cells comprise myoblasts.

4. The system of claim 1, wherein the material comprises non-living material.

5. The system of claim 1, wherein the material comprises a polymer agent.

6. The system of claim 1, wherein the material comprises a fibrin glue agent.

7. The system of claim 1, wherein the material comprises a collagen agent.

8. The system of claim 7, wherein:
the source of material comprises a first source of a first precursor material and a second source of a second precursor material;
the cardiac delivery system is adapted to couple to the first and second sources of first and second precursor materials, respectively; and
the first and second precursor materials are adapted to be mixed to form a fibrin glue.

9. The system of claim 8, wherein the cardiac delivery system is adapted to mix the first and second precursor materials prior to delivery to the location.

10. The system of claim 8, wherein:
the cardiac delivery system is adapted to deliver the first and second precursor materials to the location separately such that they are mixed at the location.

11. The system of claim 1, wherein the material is adapted to be delivered into extracellular matrix between cardiac cells at the location.

12. The system of claim 1, wherein the material is adapted to insulate against conduction via gap-junctions between cardiac cells at the location.

13. The system of claim 1, wherein:
the cardiac delivery system is adapted to deliver the material to the location along a ventricle wall of a ventricle in the patient's heart.

14. The system of claim 1, wherein:
the cardiac delivery system is adapted to deliver the material to the location along an atrial wall of an atrium in the patient's heart.

15. The system of claim 1, wherein:
the cardiac delivery system is adapted to deliver the material to the location where a pulmonary vein extends from an atrium in the patient's heart.

16. The system of claim 1, wherein the material comprises:
a first material that comprises living cells; and
a second material that is non-living and that is adapted to enhance formation of the conduction block.

17. The system of claim 16, wherein the second material comprises a polymer agent.

18. The system of claim 16, wherein the second material comprises a fibrin glue agent.

19. The system of claim 16, wherein the second material comprises a collagen agent.

20. The system of claim 19, wherein the collagen agent comprises collagen, or an analog or derivative thereof.

21. The system of claim 16, wherein the second material is adapted to enhance retention of the living cells at the location.

22. The system of claim 16, wherein the second material is adapted to insulate against conduction via gap-junctions between adjacent cells at the location.

23. The system of claim 16, wherein the living cells comprise myoblasts.

24. The system of claim 1, further comprising:
a cardiac mapping system having a mapping electrode and that is adapted to map cardiac conduction so as to locate the location.

25. The system of claim 24, wherein the mapping electrode is coupled to the cardiac delivery system.

26. The system of claim 1, further comprising an injector assembly that is adapted to inject the volume of material via the cardiac delivery system and into the location.

27. The system of claim 1, wherein the cardiac delivery system comprises:
a delivery catheter with an elongate body with a proximal end portion, a distal end portion, and a lumen extending between a proximal port along the proximal end portion and a distal port along the distal end portion;
a transeptal delivery sheath having an elongate body with proximal end portion, a distal end portion, and a delivery passageway extending between a proximal port along the proximal end portion and a distal port along the distal end portion;
wherein the transeptal delivery sheath is adapted to provide transeptal access into the left atrium of the heart via the delivery passageway;
wherein the delivery catheter is adapted to be delivered through the delivery passageway transeptally into the left atrium to thereby deliver the volume of material to the location.

28. The system of claim 1, wherein the cardiac delivery system comprises an intracardiac delivery system.

29. The system of claim 1, wherein the cardiac delivery system comprises an epicardial delivery system.

30. The system of claim 1, wherein the cardiac delivery system comprises a transvascular delivery system that is adapted to deliver the volume of material into the location through a vessel wall of a vessel associated with the cardiac tissue structure.

31. The system of claim 1, further comprising a kit adapted to prepare autologous cells as the material in an injectable form for delivery with the cardiac delivery system to the location.

32. The system of claim 1, wherein:
the cardiac delivery system comprises a delivery assembly; and
the delivery assembly is adapted to deliver the volume of material from the source and substantially along a patterned region of tissue at the location while the fluid delivery assembly is substantially secured at a position at the location.

33. The system of claim 32, wherein
the delivery assembly comprises a contact member that is adapted to substantially contact the patterned region of tissue; and
the cardiac delivery system is adapted to deliver the material substantially along the patterned region of tissue when the contact member is substantially contacting the region of tissue.

34. The system of claim 33, wherein the cardiac delivery system further comprises:
at least one needle cooperating with the contact member;
wherein the cardiac delivery system is further adapted to fluidly couple the at least one needle to the source of material and to deliver the material to the location via the at least one needle.

35. The system of claim 33, wherein the cardiac delivery system further comprises:
a plurality of needles cooperating with the contact member;
wherein the cardiac delivery system is further adapted to deliver the plurality of needles into and substantially along the patterned region of tissue and to inject the material substantially into and along the patterned region of tissue at the location via the needles.

36. The system of claim 32, wherein the delivery assembly is adapted to deliver the volume of material along an elongated pattern of tissue in the region of tissue at the location.

37. The system of claim 32, wherein the delivery assembly is adapted to deliver the volume of material along a linear pattern of tissue in the region of tissue at the location.

38. The system of claim 32, wherein the cardiac delivery system is adapted to deliver the volume of material along a curvilinear pattern of tissue in the region at the location.

39. The system of claim 32, wherein the cardiac delivery system is adapted to deliver the volume of material substantially along a circumferential region of tissue at the location so as to form a substantially circumferential conduction block at the location.

40. The system of claim 32, wherein the cardiac delivery system is adapted to deliver the volume of material along a circumferential region of tissue at the location where a pulmonary vein extends from an atrium.

41. The system of claim 40, wherein the cardiac delivery system comprises:
a contact member that is adapted to engage the circumferential region of tissue and to deliver the volume of material to the circumferential region of tissue when contacted by the contact member.

42. The system of claim 41, wherein the contact member comprises a loop-shaped member.

43. The system of claim 41, wherein the contact member comprises an expandable member.

44. The system of claim 43, wherein the expandable member comprises an inflatable balloon.

45. The system of claim 44, wherein the cardiac delivery system is adapted to deliver the material to the circumferential region of tissue when the circumferential region of tissue is engaged by the inflatable balloon.

46. A system for treating a cardiac arrhythmia in a heart of a patient, comprising:
a cardiac delivery system; and
a volume of material coupled to the cardiac delivery system;
wherein the cardiac delivery system is adapted to deliver the volume of material to a region of tissue at a location associated with the cardiac arrhythmia in the patient's heart that includes cardiac cells;
wherein the material is non-living and is characterized as being non-ablative with respect to cardiac cells;
wherein the material is further adapted to form a conduction block along the region of tissue at the location; and
wherein the system does not include living cells.

47. The system of claim 46, wherein the cardiac delivery system comprises:
an intracardiac delivery system that is adapted to provide intracardiac delivery of the volume of material to the location via at least one of the cardiac chambers.

48. The system of claim 46, wherein the cardiac delivery system comprises:
an epicardial cardiac delivery system.

49. The system of claim 46, wherein the cardiac delivery system comprises:
a transvenous cardiac delivery system.

50. The system of claim 46, wherein the cardiac delivery system is adapted to deliver the volume of material substantially along a patterned region of tissue at the location.

51. The system of claim 50, wherein the cardiac delivery system comprises:
a contact member that is adapted to substantially contact the patterned region of tissue; and
wherein the cardiac delivery system is adapted to deliver the material substantially along the patterned region of tissue when the contact member is substantially contacting the region of tissue.

52. The system of claim 51, wherein
the contact member comprises an elongated member that is adapted to contact an elongated region of tissue at the location; and
the cardiac delivery system is adapted to deliver the material to the elongated region of tissue when the contact member is substantially contacting the elongated region of tissue.

53. The system of claim 51, wherein:
the contact member is adapted to engage a substantially circumferential region of tissue at the location; and
the cardiac delivery system is adapted to deliver the material to the circumferential region of tissue when the contact member is substantially contacting the circumferential region of tissue.

54. The system of claim 53, wherein the contact member comprises a loop-shaped member.

55. The system of claim 53, wherein the contact member comprises an expandable member.

56. The system of claim 55, wherein the expandable member comprises an inflatable balloon.

57. The system of claim 56, wherein the cardiac delivery system is adapted to deliver the material to the circumferential region of tissue when the circumferential region of tissue is engaged by the inflatable balloon.

58. The system of claim 51, wherein the cardiac delivery system further comprises:
a plurality of needles cooperating with the contact member;
wherein the cardiac delivery system is further adapted to deliver the plurality of needles into and substantially along the patterned region of tissue and to inject the material substantially into and along the patterned region of tissue at the location via the needles.

59. The system of claim 56, wherein the material comprises an injectable polymer agent.

60. The system of claim 59, wherein the injectable polymer agent comprises a fibrin glue agent.

61. The system of claim 59, wherein the injectable polymer agent comprises a collagen agent.

62. The system of claim 51, wherein the collagen agent comprises collagen, or an analog or derivative thereof.

63. The system of claim 59, wherein the injectable polymer agent comprises:
a first precursor material;
a second precursor material; and
wherein the first and second precursor materials are adapted to be mixed to form a polymerized agent.

64. The system of claim 63, wherein the cardiac delivery system is adapted to mix the first and second precursor materials to form the polymerized agent before delivering the polymerized agent to the location.

65. The system of claim 63, wherein the cardiac delivery system is adapted to deliver the first and second precursor materials to the location separately such that they mix and form the polymerized agent at the location.

66. The system of claim 46, wherein the cardiac delivery system comprises at least one needle.

67. The system of claim 46, wherein the cardiac delivery system comprises:
a catheter having an elongate body with a proximal end portion, a distal end portion, and at least one lumen extending between a proximal port located along the proximal end portion and a distal port located along the distal end portion; and
wherein the proximal port is adapted to couple to a source that contains at least a part of the material.

68. The system of claim 67, wherein the catheter further comprises:
at least one mapping electrode located along the distal end portion; and
wherein the at least one electrode is adapted to be coupled to a monitoring system to monitor electrical signals in the heart via the electrode so as to identify the location for delivery of the material to thereby form the conduction block.

69. A system for treating a cardiac arrhythmia in a heart of a patient, comprising:
a cardiac delivery system with a contact member and also with a plurality of needles cooperating with the contact member;
a source of material that is adapted to be coupled to the cardiac delivery system;
wherein the contact member is adapted to be delivered to the location and to substantially contact a patterned region of tissue at a location associated with the arrhythmia and that includes cardiac cells;
wherein the plurality of needles are adapted to be inserted into and substantially along the patterned region of tissue when the contact member is contacted with the patterned region of tissue;
wherein the cardiac delivery system is adapted to be coupled to the source of material and to deliver a volume of the material from the source into and substantially along the patterned region of tissue via the plurality of needles; and
wherein the material is substantially non-ablative to the cardiac tissue and is adapted to form a substantially non-ablative conduction block along the patterned region of tissue at the location.

70. A system for treating a cardiac arrhythmia in a patient, comprising:
a cardiac delivery system;
a source of material coupled to the cardiac delivery system;
wherein the cardiac delivery system comprises an expandable member;
wherein the source of material comprises living cells; and
wherein the cardiac delivery system is adapted to deliver the living cells from the source to a region of tissue at a location associated with the cardiac arrhythmia and that includes cardiac cells; and
wherein the material is adapted to form a conduction block in the region.

71. A system for treating a cardiac arrhythmia in a patient, comprising:
a cardiac delivery device with a guidewire tracking member;
a guidewire adapted to slideably engage the guidewire tracking member;
a source of material coupled to the cardiac delivery device;
wherein the cardiac delivery device is adapted to track over the guidewire to a location associated with the cardiac arrhythmia;
wherein the cardiac delivery device is further adapted to deliver a volume of the material from the source into a region of tissue at the location and that includes cardiac cells;
wherein the material is characterized as being substantially non-ablative to cardiac cells; and
wherein the material is adapted to form a conduction block in the region.

72. A system for treating a cardiac arrhythmia in a patient, comprising:
a cardiac delivery device;
a source of material coupled to the cardiac delivery device;
wherein the cardiac delivery device is adapted to deliver a volume of the material from the source and to a circumferential region of tissue at a location where a pulmonary vein extends from an atrium while the cardiac delivery device is substantially secured at a position at the location;
wherein the cardiac delivery device is further adapted to allow blood to perfuse downstream across the location while delivering the material into the circumferential region of tissue;
wherein the material is characterized as being substantially non-ablative to cardiac tissue; and
wherein the material is adapted to form a conduction block in the circumferential region of tissue.

73. A method for treating a cardiac arrhythmia in a heart of a patient, comprising:
delivering a volume of material that is non-ablative to cardiac cells into a region of tissue at a location associated with the cardiac arrhythmia in the patient's heart and that includes cardiac cells;
forming a conduction block in the region with the material.

74. The method of claim 73, wherein the material delivery to the region further comprises:
delivering a non-living material to the region; and
whereby the conduction block is formed at the location at least in part with the non-living material.

75. The method of claim 74, further comprising:
insulating conduction via gap-junctions between adjacent cardiac tissue with the material delivered to the region; and
whereby the conduction block is formed at least in part by insulating the conduction with the material.

76. The method of claim 74, wherein the material delivery to the region of tissue at the location further comprises:
delivering a polymer agent to the region of tissue at the location.

77. The method of claim 76, wherein the material delivery to the region of tissue at the location further comprises:
delivering a collagen agent to the region of tissue at the location.

78. The method of claim 77, wherein the collagen agent comprises collagen, or an analog or derivative thereof.

79. The method of claim 74, wherein the material delivery to the region of tissue at the location further comprises:
delivering a fibrin glue agent to the region of tissue at the location.

80. The method of claim 79, further comprising:
mixing first and second precursor materials within the body of the patient to form a polymerized fibrin glue in situ.

81. The method of claim 74, wherein the material delivery to the region of tissue at the location further comprises:
delivering living cells to the region of tissue at the location.

82. The method of claim 81, wherein the delivery of living cells to the region of tissue at the location further comprises:
delivering myoblasts to the region of tissue at the location.

83. The method of claim 74, wherein the material delivery to the region of tissue at the location further comprises:
delivering the material to the region of tissue at the location along a ventricular wall of a ventricle of the patient's heart.

84. The method of claim 74, wherein the material delivery to the region of tissue at the location further comprises:
delivering the material to the region of tissue at the location along an atrial wall of an atrium of the patient's heart.

85. The method of claim 74, wherein the material delivery to the region of tissue at the location further comprises:
delivering the material to the region of tissue at the location where a pulmonary vein extends from an atrium.

86. The method of claim 74, wherein the material delivery to the region of tissue at the location further comprises:
delivering the material along a patterned region of tissue at the location.

87. The method of claim 86, wherein delivering the material along the patterned region of tissue comprises:
delivering the material along an elongated region of tissue at the location.

88. The method of claim 86, wherein delivering the material along the patterned region of tissue comprises:
delivering the material along a substantially circumferential region of tissue at the location.

89. The method of claim 88, further comprising:
contacting the patterned region of tissue at the location with a contact member; and
delivering the volume of material to the patterned region of tissue while substantially contacting the patterned region of tissue with the contact member.

90. The method of claim 74, further comprising:
anchoring a delivery device with an anchor at a position associated with the location;
delivering the material to the region of tissue at the location while the anchor is anchored at the position.

91. A method for treating a cardiac arrhythmia in a heart of a patient, comprising:
forming a conduction block at a location associated with the patient's heart that includes cardiac cells by delivering living cells to the location.

92. The method of claim 91, further comprising:
forming the conduction block at a location where a pulmonary vein extends from an atrium by delivering skeletal myoblasts to the location.

93. A method for assembling a cardiac arrhythmia treatment system from a plurality of cardiac delivery systems, wherein each cardiac delivery system is adapted to deliver a volume of material either along a unique pattern of cardiac tissue, or at a unique location associated with the heart of a patient, the method comprising:
choosing a cardiac delivery system from the plurality of cardiac delivery systems based upon at least one known patterned region of tissue and location where a conduction block is to be formed;
coupling a volume of material to the cardiac delivery system;
wherein the chosen cardiac delivery system is adapted to deliver the volume of injectable material into and along the patterned region of tissue at the location;
wherein the material is adapted to be injected by the cardiac delivery system into and along the patterned region of tissue at the location; and
wherein the material is substantially non-ablative to cardiac tissue and is adapted to form a substantially non-ablative conduction block when delivered into and along the patterned region of tissue at the location.

94. A method for treating a cardiac condition in a patient, comprising:
tracking a delivery device over a guidewire to a location associated with the heart;
delivering a volume of living cells to a region of tissue that includes cardiac cells with the delivery device.

95. A method for assembling a cardiac treatment system, comprising:
coupling a source of living cells to a delivery catheter that comprises an expandable member.

96. A method for assembling a cardiac treatment system, comprising:
coupling a source of living cells to a delivery catheter with a guidewire disposed within a guidewire tracking member of the delivery catheter.
